SEQUENCE LISTING (25 sequences)

SEQ ID NO: 1 - DNA, 3064 bp, Homo sapiens
  variation (184)..(184): polymorphism
gaggaagccgatttccctggtgtggtccaggcacctgcgagacaatgtgcattttgttttaaaaagcagccacttgtgatttgcaacaaaggttatcagtgtaaatgggaaaggcttaattgcccaagaaatacaggttcatgttatattcatttgaatccaggcagccaaggtttacaaatgnaagtgcattattcctctgggtgtgtttccctgtccaatatttacattaaccattttaataatcatgtgggcttaaaagatttgccagaaatggggggtagggtggagagggaaagacaaatgattaaatcacatcaaatcttgggacaagtaagttcctattaattttgaagttaattttgaaattttgctttaagatgaatcaacaaatactcttatgcagcatgaatagtggcacttaagatatattttgcacgggttctttggacccacatcaaacagagaagctgcctgtgaaacactgacagatactaatagatctcaaataaatgtaggtagtgtctctgcctcctaatagcaatcttgtagattccattgcaattctagtggttaattcctggactggcatattttacaagaatgggacattttattccttctctcctaaagttaccctatcaaatgtatattgtccaaaatagggacattttaaaattcacttagcttgaaaaactaaacaagccaattctctgaaatctcgagcaaaaatatttagtaaagcaaatattagtattcaaaattaaaactttatttgcttccccataagtgaacagctttgttagcactgtctgacatcattgcttgttaacttaagaactgataggtttttttttttttttttttcagatattctgatggcaaaacaagtggaagaaaagaggaagcatgactgcagatcagatcagttctctttgtggattatattttcagtaaaatgtatggatctatcttttccttgttcttatatctagatcatgagacttgactgaggctgtatncttatcctccatccatctatggcgaactatagccatgcagctgacaacattttgcaaaatctctcgcctctaacagcctttctgaaactgacttccttgggtttcataataggagtcagcgtggtgggcaacctcctgatctccattttgctagtgaaagataagaccttgcatagagcaccttactacttcctgttggatctttgctgttcagatatcctcagatctgcaatttgtttcccatttgtgttcaactctgtcaaaaatggntctacctggacttatgggactctgacttgcaaagtgattgcctttctgggggttttgtcctgtttccacactgctttcatgctcttctgcatcagtgtcaccagatanttagctatcgcccatcaccgcttctatacaaagaggctgaccttttggacgtgtctggctgtgatctgtatggtgtggactctgtctgtggccatggcatttcccccggttttagacgtgggcacttactcattcattagggaggaagatcaatgcaccttccaacaccgctccttcagggctaatgattccttaggatttatgctgcttcttgctctcatcctcctagccacacagcttgtctacctcaagctgatatttttcgtccacgatcgaagaaaaatgaagccagtccagtttgtagcagcagtcagccagaactggacttttcatggtcctggagccagtggccaggcagctgccaattggctagcaggatttggaaggggtcccacaccacccaccttgctgggcatcaggcaaaatgcaaacaccacaggcagaagaaggctattggtcttagacgagttcaaaatggagaaaagaatcagcagaatgttctatataatgacttttctgtttctaaccttgtggggcccctacctggtggcctgttattggagagtttttgcaagagggcctgtagtaccagggggatttctaacagctgctgtctggatgagttttgcccaagcaggaatcaatccttttgtctgcattttctcaaacagggagctgaggcgctgtttcagcacaacccttctttactgcagaaaatccaggttaccaagggaaccttactgtgttatatgagggagcatctgtaaatctttagccttgtgaaaactaaccttctctgctgagcaattgtggcccatagccatattttgagaagaaattcaagaatggaatcagcagttttaaggatttgggcaacattctgcagtctttgcaanagttcacctataatcctattttaaatctcagagtgatcctgctgactgccagcaaaggtttgtaattaagaanggactgaaccactgccctaagtttctttatgtggtcaaaaactagataatgaaagtagcaggtgctaagtatcagtgctaaatgctctgtatgtcactacatatgaaaaaacatcaaaaaacaattagcattggacatcttaataaattaagttgacatgaggtaaatgtgttgataaaaactaattttagaagtttgaagactttaaaacatttcatactactattgttttgcaaagactaaaatatttggggacttaaagtactgtaatccactaaagacgtgccaatgaattattggaatatcacactttaaaaaccgccttgtaagttctggggagcattccaaagcagtntattggttccaattagagtttactttttttgtattaatacattgctatttctaaataccactttcctcatctactagtaagattgctagcattgaactgtattatgtggtttttgttgatttggtataaagtttttccaattcatttatattttacaaatgctagatattggtctgggaggcaacattaatggtaccagcctgtcacaactgagcagttctaataatgcagaataaatacatgttgccttaaagggttatctagtatccttcatcttatttagcactggagcaaatagccaagggaaatcaaatcagtaactggtcatggtcatgcatct

SEQ ID NO: 2 - DNA, 20 bp, Artificial Sequence
  oligonucleotide
gaggaagccgatttccctgg

SEQ ID NO: 3 - DNA, 21 bp, Artificial Sequence
  oligonucleotide
cacaaatgggaaacaaattgc

SEQ ID NO: 4 - DNA, 27 bp, Artificial Sequence
  oligonucleotide
agctatggcgaactatagccatgcagc

SEQ ID NO: 5 - DNA, 20 bp, Artificial Sequence
  oligonucleotide
agatgcatgaccatgaccag

SEQ ID NO: 6 - DNA, 17 bp, Artificial Sequence
  oligonucleotide
atgcacttccatttgta

SEQ ID NO: 7 - DNA, 15 bp, Artificial Sequence
  oligonucleotide
atgcacttgcatttg

SEQ ID NO: 8 - DNA, 16 bp, Artificial Sequence
  oligonucleotide
aggctgtatccttatc

SEQ ID NO: 9 - DNA, 16 bp, Artificial Sequence
  oligonucleotide
aggctgtatacttatc

SEQ ID NO: 10 - DNA, 15 bp, Artificial Sequence
  oligonucleotide
ccaggtagagccatt

SEQ ID NO: 11 - DNA, 16 bp, Artificial Sequence
  oligonucleotide
tccaggtagaaccatt

SEQ ID NO: 12 - DNA, 17 bp, Artificial Sequence
  oligonucleotide
cgatagctaagtatctg

SEQ ID NO: 13 - DNA, 17 bp, Artificial Sequence
  oligonucleotide
cgatagctaaatatctg

SEQ ID NO: 14 - DNA, 17 bp, Artificial Sequence
  oligonucleotide
caaagcagtatattggt

SEQ ID NO: 15 - DNA, 17 bp, Artificial Sequence
  oligonucleotide
caaagcagtgtattggt

SEQ ID NO: 16 - DNA, 32 bp, Artificial Sequence
  oligonucleotide
aaatacaggttcatgttatattcatttgaatc

SEQ ID NO: 17 - DNA, 28 bp, Artificial Sequence
  oligonucleotide
ttaatgtaaatattggacagggaaacac

SEQ ID NO: 18 - DNA, 32 bp, Artificial Sequence
  oligonucleotide
atggatctatcttttccttgttcttatatcta

SEQ ID NO: 19 - DNA, 23 bp, Artificial Sequence
  oligonucleotide
tgtcagctgcatggctatagttc

SEQ ID NO: 20 - DNA, 24 bp, Artificial Sequence
  oligonucleotide
ttgtttcccatttgtgttcaactc

SEQ ID NO: 21 - DNA, 24 bp, Artificial Sequence
  oligonucleotide
agaaaggcaatcactttgcaagtc

SEQ ID NO: 22 - DNA, 22 bp, Artificial Sequence
  oligonucleotide
tcctgtttccacactgctttca

SEQ ID NO: 23 - DNA, 20 bp, Artificial Sequence
  oligonucleotide
agccagacacgtccaaaagg

SEQ ID NO: 24 - DNA, 26 bp, Artificial Sequence
  oligonucleotide
gtgccaatgaattattggaatatcac

SEQ ID NO: 25 - DNA, 29 bp, Artificial Sequence
  oligonucleotide
gaggaaagtggtatttagaaatagcaatg

(19) United States
(12) Patent Application Publication
(10) Pub. No.: US 2003/0224365 A1
(43) Pub. Date: Dec. 4, 2003

(54) Single nucleotide polymorphisms diagnostic for schizophrenia

(76) Inventors: Linda Susan Wood, Portage, MI (US); Kathleen A. Schellin, Kalamazoo, MI (US); Chi-Hse Teng, Portage, MI (US)

Correspondence Address: PHARMACIA & UPJOHN, 301 HENRIETTA ST, 0228-32-LAW, KALAMAZOO, MI, 49007, US

(21) Appl. No.: 10/126,063

(22) Filed: Apr. 19, 2002

Related U.S. Application Data
(60) Provisional application No. 60/286,400, filed on Apr. 24, 2001.

(51) Int. Cl. C12Q 1/68; C07H 21/04; C07K 14/705; C12P 21/02; C12N 5/06

(52) U.S. Cl. 435/6; 435/69.1; 435/320.1; 435/325; 530/350; 536/23.2

(57) ABSTRACT

The invention provides nucleic acid segments of the human G protein coupled receptor Con-202 gene including polymorphic sites. Allele specific primers and probes hybridizing to regions flanking these sites are also provided. The invention also provides methods for determining the genetic risk of developing schizophrenia or diagnosing schizophrenia.

### CROSS REFERENCE TO RELATED APPLICATION

This application claims the benefit of the following provisional application: Application Serial No. 60/286,400 filed Apr. 24, 2001 under 35 U.S.C 119(e)(1).

### FIELD OF THE INVENTION

The invention provides nucleic acid segments of a human G-protein coupled receptor Con-202 including polymorphic sites. The invention also provides methods for determining the genetic risk of developing schizophrenia or diagnosing schizophrenia.

### BACKGROUND

Single Nucleotide Polymorphisms

All organisms undergo periodic mutation in the course of their evolution and thus generate variant forms of progenitor sequences (Gusella, Ann. Rev. Biochem. 55, 831-854 (1986)). The variant form may or may not confer an evolutionary advantage relative to a progenitor form. The variant form may be neutral. In some instances, a variant form is lethal and is not transmitted to further generations of the organism. In other instances, a variant form confers an evolutionary advantage to the species and is eventually incorporated into the DNA of many or most members of the species and effectively becomes the progenitor form. In many instances, both progenitor and variant form(s) survive and co-exist in a species population. This coexistence of multiple forms of a sequence gives rise to polymorphisms.

Several different types of polymorphism have been reported. A restriction fragment length polymorphism (RFLP) means a variation in DNA sequence that alters the length of a restriction fragment as described in Botstein et al., Am. J. Hum. Genet. 32, 314-331 (1980). The restriction fragment length polymorphism may create or delete a restriction site, thus changing the length of the restriction fragment. RFLPs have been widely used in human and animal genetic analyses (see U.S. Pat. No. 5,856,104, Jan. 5, 1999, Chee, et al, WO 90/13668; WO90/11369; Donis-Keller, Cell 51, 319-337 (1987); Lander et al., Genetics 121, 85-99 (1989)). When a heritable trait can be linked to a particular RFLP, the presence of the RFLP in an individual can be used to predict the likelihood that the animal will also exhibit the trait.

Other polymorphisms take the form of short tandem repeats (STRs) that include tandem di-, tri- and tetranucleotide repeated motifs. These tandem repeats are also referred to as variable number tandem repeat (VNTR) polymorphisms. VNTRs have been used in identity and paternity analysis (U.S. Pat. No. 5,075,217; Armour et al., FEBS Lett. 307, 113-115 (1992); Horn et al., WO 91/14003; Jeffreys, EP 370,719), and in a large number of genetic mapping studies.

Some other polymorphisms take the form of single nucleotide variations between individuals of the same species. Such polymorphisms are far more frequent than RFLPS, STRs and VNTRs. Although it should be recognized that a single nucleotide polymorphism may also result in a RFLP because a single nucleotide change can also result in the creation or destruction of a restriction enzyme site. Some single nucleotide polymorphisms occur in protein-coding sequences, in which case, one of the polymorphic forms may give rise to the expression of a defective or other variant protein and, potentially, a genetic disease. Examples of genes, in which polymorphisms within coding sequences give rise to genetic disease include beta-globin (sickle cell anemia) and CFTR (cystic fibrosis). Other single nucleotide polymorphisms occur in noncoding regions. Some of these polymorphisms may also result in defective protein expression (e.g., as a result of defective splicing). Other single nucleotide polymorphisms have no phenotypic effects but still may be genetically linked to a phenotypic effect.

The greater frequency and uniformity of single nucleotide polymorphisms means that there is a greater probability that such a polymorphism will be found in close proximity to a genetic locus of interest than would be the case for other polymorphisms. Also, the different forms of characterized single nucleotide polymorphisms are often easier to distinguish that other types of polymorphism (e.g., by use of assays employing allele-specific hybridization probes or primers). In a disease such as schizophrenia in which multiple gene products play a role in the analysis of the disease, SNPs show particular promise as a research tool and they may also be valuable diagnostic tools.

Schizophrenia

Schizophrenia is a devastating neuropsychiatric disorder which affects approximately 1% of the population and results in serious disruption in the lives of afflicted individuals and their families. Common symptoms include delusions, conceptual disorganizations and visual or auditory hallucinations, as well as changes in affective behavior. A number of scales for the rating of symptoms and methods for ascertaining the diagnosis have been developed, including the DSM classification by the American Psychiatric Association (Diagnostic and Statistical Manual of Mental Disorders Third and Fourth Editions), which have attempted to refine the accuracy of clinical diagnosis. However, it is likely that similar symptoms can result from several underlying abnormalities, and diagnosis relying solely on clinical symptoms is difficult and controversial, as well as subjective, time-consuming and costly. There is a pressing need therefore, for new methods of diagnosing or predicting predisposition to develop schizophrenia.

### LITERATURE CITED

U.S. Pat. Nos.

1. U.S. Pat. No. 5,856,104 Polymorphisms in the glucose-6 phosphate dehydrogenase locus

2. U.S. Pat. No. 5,075,217 Length polymorphisms in (dC-dA)ₙ(dG-dT)ₙ sequences

3. U.S. patent application Ser. No. 09/427,653 filed on Oct. 27, 1999

4. U.S. patent application Ser. No. 09/698,419 filed Oct. 27, 2000

5 U.S. Pat. No. 4,683,202 Process for amplifying nucleic acid sequences

6. U.S. Pat. No. 5, 185, 444 Uncharged morpolino-based polymers having phosphorous containing chiral intersubunit linkages

7. U.S. Pat. No. 5, 034, 506 Uncharged morpholino-based polymers having achiral intersubunit linkages

8. U.S. Pat. No. 5, 142, 047 Uncharged polynucleotide-binding polymers

9. U.S. Pat. No 5, 424, 186 Very large scale immobilized polymer synthesis

10. U.S. Pat. No. 6, 071,722 Nucleic Acids encoding a G-protein Coupled 7TM Receptor (AXOR-1)

Foreign Patent Documents

1. WO90/13668 Method for Genetic Analysis of a Nucleic Acid Sample

2. WO90/11369 Solid Phase Diagnosis of Medical Conditions

3. WO91/14003 Characterization and Analysis of Polymorphic VNTR Loci

4. EP370719 Extended nucleotide sequences

5. WO9504064 Polynucleotide Decoys that Inhibit MHC-II Expression and Uses Thereof

6. EP235726 Rapid detection of nucleic acid sequences in a sample by labeling the sample

7. WO8911548 Immobilized Sequence Specific Probes

8. WO9322456 Detection of Gene Sequences in Biological Fluids

9. WO9820165 Biallelic Markers

10. WO9210092 Very large scale immobilized polymer synthesis

11. WO9511995 Arrays of Nucleic Acid Probes on Biological Chips

12. WO0155387 Nucleic Acids, Proteins, andAntibodies

13. WO0107612 Receptors and Associated Proteins

14. WO0185791 Nucleic Acid Sequences for Novel GPCRS

15. WO0131014 G Protein Coupled Receptors Expressed in Brain

16. WO0132865 Human GPR27-Like G-Protein Coupled Receptor Polypeptide and Polynucleotide Sequences

17. WO0164835 Novel Nucleic Acids and Polypeptides

Books

1. Diagnostic and Statistical Manual of Mental Disorders (4^{th} Edition), 273-316, 1994

2. DNA Sequencing” in Sambrook et al. (eds.), Molecular Cloning: A Laboratory Manual (Second Edition), Plainview, N.Y.: Cold Spring Harbor Laboratory Press (1989)

3. Biochemistry, 4^{th} edition, Stryer, L., 1995

4. PCR Technology: Principles and Applications for DNA Amplification (ed. H. A. Erlich, Freeman Press, N.Y., N.Y., 1992);

5. PCR Protocols: A Guide to Methods and Applications (eds. Innis, et al., Acadernic Press, San Diego, Calif., 1990); Mattila et al., Nucleic Acids Res. 19, 4967 (1991);

6. PCR Methods and Applications Eckert et al.,1, 17 (1991);

7. PCR (eds. McPherson et al., IRL Press, Oxford)

Public Database Entries

1. Genbank AC073346

2. Genbank AL161959

3. Genbank AF250237

4. Genbank AF254416

5. Genbank AC097542

6. Genbank AF203907

7. Genbank AB040803

8. Genbank AL540051

9. Genbank BF41462

10.Genbank R35286

11. Genbank BI734823

12. Genbank AL540052

13.Genbank BB647727

14. Genbank AA883367

Journal Articles

1. Ajioka, R. S., et al., Haplotype analysis of hemochromatosis: evaluation of different linkage—disequilibrium approaches and evolution of disease chromosomes. Am J Hum Genet, 1997. 60(6): p. 1439-47.

2. Armour, J. A. and A. J. Jeffreys, Recent advances in minisatellite biology. FEBS Lett, 1992. 307(1): p. 113-5.

3. Botstein, D., et al., Construction of a genetic linkage map in man using restriction fragment length polymorphisms. Am J Hum Genet, 1980. 32(3): p. 314-31.

4. Clark, A. G., Inference of haplotypes from PCR-amplified samples of diploid populations. Mol Biol Evol, 1990. 7(2): p. 111-22.

5. Clayton, D., A generalization of the transmission/disequilibrium test for uncertain-haplotype transmission. Am J Hum Genet, 1999. 65(4): p. 1170-7.

6. Donis-Keller, H., et al., A genetic linkage map of the human genome. Cell, 1987. 51(2): p. 319-37.

7. Excoffier, L. and M. Slatkin, Maximum-likelihood estimation of molecular haplotype frequencies in a diploid population. Mol Biol Evol, 1995. 12(5): p. 921-7.

8. Germer, S. and R. Higuchi, Single-tube genotyping without oligonucleotide probes. Genome Res, 1999. 9(1): p. 72-8.

9. Gibbs, R. A., P. N. Nguyen, and C. T. Caskey, Detection of single DNA base differences by competitive oligonucleotide priming. Nucleic Acids Res, 1989. 17(7): p. 2437-48.

10. Guatelli, J. C., et al., Isothermal, in vitro amplification of nucleic acids by a multienzyme reaction modeled after retroviral replication. Proc Natl Acad Sci USA, 1990. 87(5): p. 1874-8.

11. Gusella, J. F., DNA polymorphism and human disease. Annu Rev Biochem, 1986.55: p. 831-54.

12. Hacia, J. G., et al., Detection of heterozygous mutations in BRCA1 using high density oligonucleotide arrays and two-colour fluorescence analysis. Nat Genet, 1996. 14(4): p. 441-7.

13. Hawley, M. E. and K. K. Kidd, HAPLO: a program using the EM algorithm to estimate the frequencies of multi-site haplotypes. J Hered, 1995. 86(5): p. 409-11.

14. Howard, T. D., E. R. Bleecker, and O. C. Stine, Fluorescent allele-specific PCR (FAS-PCR) improves the reliability of single nucleotide polymorphism screening. Biotechniques, 1999. 26(3): p. 380-1.

15. Kozal, M. J., et al., Extensive polymorphisms observed in HIV-1 clade B protease gene using high-density oligonucleotide arrays. Nat Med, 1996. 2(7): p. 753-9.

16. Kwoh, D. Y., et al., Transcription-based amplification system and detection of amplified human immunodeficiency virus type I with a bead-based sandwich hybridization format. Proc Natl Acad Sci USA, 1989. 86(4): p. 1173-7.

17. Landegren, U., et al., A ligase-mediated gene detection technique. Science, 1988. 241(4869): p. 1077-80.

18. Landegren, U., M. Nilsson, and P. Y. Kwok, Reading bits of genetic information: methods for single-nucleotide polymorphism analysis. Genome Res, 1998. 8(8): p. 769-76.

19. Lander, E. S. and D. Botstein, Mapping mendelian factors underlying quantitative traits using RFLP linkage maps. Genetics, 1989. 121(1): p. 185-99.

20. Lander, E. S. and N. J. Schork, Genetic dissection of complex traits. Science, 1994. 265(5181): p. 2037-48.

21. Livak, K. J., J. Marmaro, and J. A. Todd, Towards fully automated genome-wide polymorphism screening. Nat Genet, 1995. 9(4): p. 341-2.

22. Mattila, P., et al., Fidelity of DNA synthesis by the Thermococcus litoralis DNA polymerase—an extremely heat stable enzyme with proofreading activity. Nucleic Acids Res, 1991. 19(18): p. 4967-73.

23. Newton, C. R., et al., Analysis of any point mutation in DNA. The amplification refractory mutation system (ARMS). Nucleic Acids Res, 1989. 17(7): p. 2503-16.

24. Nickerson, D. A., V. O. Tobe, and S. L. Taylor, PolyPhred: automating the detection and genotyping of single nucleotide substitutions using fluorescence-based resequencing. Nucleic Acids Res, 1997. 25(14): p. 2745-51.

25. Nielsen, P. E., et al., Sequence-selective recognition of DNA by strand displacement with a thymine-substituted polyamide. Science, 1991. 254(5037): p. 1497-500.

26. Orita, M., et al., Detection of polymorphisms of human DNA by gel electrophoresis as single-strand conformation polymorphisms. Proc Natl Acad Sci USA, 1989. 86(8): p. 2766-70.

27. Perlin, M. W., et al., Toward fully automated genotyping: allele assignment, pedigree construction, phase determination, and recombination detection in Duchenne muscular dystrophy. Am J Hum Genet, 1994. 55(4): p. 777-87.

28. Ruano, G., K. K. Kidd, and J. C. Stephens, Haplotype of multiple polymorphisms resolved by enzymatic amplification of single DNA molecules. Proc Natl Acad Sci USA, 1990. 87(16): p. 6296-300.

29. Saiki, R. K., et al., Analysis of enzymatically amplified beta-globin and HLA-DQ alpha DNA with allele-specific oligonucleotide probes. Nature, 1986. 324(6093): p. 163-6.

30. Sarkar, G. and S. S. Sommer, Haplotyping by double PCR amplification of specific alleles. Biotechniques, 1991. 10(4): p. 436, 438, 440.

31. Schaid, D. J., General score tests for associations of genetic markers with disease using cases and their parents. Genet Epidemiol, 1996. 13(5): p. 423-49.

32. Shoemaker, D. D., et al., Quantitative phenotypic analysis of yeast deletion mutants using a highly parallel molecular bar-coding strategy. Nat Genet, 1996. 14(4): p. 450-6.

33. Spielman, R. S., R. E. McGinnis, and W. J. Ewens, Transmission test for linkage disequilibrium: the insulin gene region and insulin-dependent diabetes mellitus (IDDM). Am J Hum Genet, 1993. 52(3): p. 506-16.

34. Spielman, R. S. and W. J. Ewens, A sibship test for linkage in the presence of association: the sib transmission/disequilibrium test. Am J Hum Genet, 1998. 62(2): p. 450-8.

35. Tyagi, S., D. P. Bratu, and F. R. Kramer, Multicolor molecular beacons for allele discrimination. Nat Biotechnol, 1998. 16(1): p. 49-53.

36. Wu, D. Y. and R. B. Wallace, The ligation amplification reaction (LAR)—amplification of specific DNA sequences using sequential rounds of template-dependent ligation. Genomics, 1989. 4(4): p. 560-9.

37. Wu, D. Y., et al., Allele-specific enzymatic amplification of beta-globin genomic DNA for diagnosis of sickle cell anemia. Proc Natl Acad Sci USA, 1989. 86(8): p. 2757-60.

38. Zhao, L. P., et al., Mapping of complex traits by single-nucleotide polymorphisms. Am J Hum Genet, 1998. 63(1): p. 225-40.

### SUMMARY OF THE INVENTION

The invention is based on the discovery of a set of schizophrenia related polymorphic markers. These markers are located in the coding region as well as the non-coding region of the gene for the G-protein coupled receptor (GPCR) we have designated Con-202. The coding and relevant non-coding regions of Con-202 are set forth below. Intron sequence is designated by small letters and exon sequence is delineated in capital letters. The polymorphisms are in bold type and underlined.

| | |
|---|---|
| 1 | gaggaagccg atttccctgg tgtggtccag gcacctgcga gacaatgtgc |
| 51 | attttgtttt aaaaagcagc cacttgtgat ttgcaacaaa ggttatcagt |
| 101 | gtaaatggga aaggcttaat tgcccaagaa atacaggttc atgttatatt |
| 151 | catttgaatc caggcagcca aggtttacaa atgg/caagtgc attattcctc |
| 201 | tgggtgtgtt tccctgtcca atatttacat taaccatttt aataatcatg |
| 251 | tgggcttaaa agatttgcca gaaatggggg gtagggtgga gagggaaaga |
| 301 | caaatgatta aatcacatca aatcttggga caagtaagtt cctattaatt |
| 351 | ttgaagttaa ttttgaaatt ttgctttaag atgaatcaac aaatactctt |
| 401 | atgcagcatg aatagtggca cttaagatat attttgcacg ggttctttgg |
| 451 | acccacatca aacagagaag ctgcctgtga aacactgaca gatactaata |
| 501 | gatctcaaat aaatgtaggt agtgtctctg cctcctaata gcaatcttgt |
| 551 | agattccatt gcaattctag tggttaattc ctggactggc atattttaca |
| 601 | agaatgggac attttattcc ttctctccta aagttaccct atcaaatgta |
| 651 | tattgtccaa aatagggaca ttttaaaatt cacttagctt gaaaaactaa |
| 701 | acaagccaat tctctgaaat ctcgagcaaa aatatttagt aaagcaaata |
| 751 | ttagtattca aaattaaaac tttatttgct tccccataag tgaacagctt |
| 801 | tgttagcact gtctgacatc attgcttgtt aacttaagaa ctgataggtt |
| 851 | tttttttttt tttttttcag ATATTCTGAT GGCAAAACAA GTGGAAGAAA |
| 901 | AGAGGAAGCA TGACTGCAGA TCAGATCAGT TCTCTTTGTG GATTATATTT |
| 951 | TCAGTAAAAT GTATGGATCT ATCTTTTCCT TGTTCTTATA TCTAGATCAT |
| 1001 | GAGACTTGAC TGAGGCTGTA TC/ACTTATCCT CCATCCATCT [symbol] GCGAACT |
| 1051 | ATAGCCATGC AGCTGACAAC ATTTTGCAAA ATCTCTCGCC TCTAACAGCC |
| 1101 | TTTCTGAAAC TGACTTCCTT GGGTTTCATA ATAGGAGTCA GCGTGGTGGG |
| 1151 | CAACCTCCTG ATCTCCATTT TGCTAGTGAA AGATAAGACC TTGCATAGAG |
| 1201 | CACCTTACTA CTTCCTGTTG GATCTTTGCT GTTCAGATAT CCTCAGATCT |
| 1251 | GCAATTTGTT TCCCATTTGT GTTCAACTCT GTCAAAAATG GC/TTCTACCTG |
| 1301 | GACTTATGGG ACTCTGACTT GCAAAGTGAT TGCCTTTCTG GGGGTTTTGT |
| 1351 | CCTGTTTCCA CACTGCTTTC ATGCTCTTCT GCATCAGTGT CACCAGATAC/T |
| 1401 | TTAGCTATCG CCCATCACCG CTTCTATACA AAGAGGCTGA CCTTTTGGAC |
| 1451 | GTGTCTGGCT GTGATCTGTA TGGTGTGGAC TCTGTCTGTG GCCATGGCAT |
| 1501 | TTCCCCCGGT TTTAGACGTG GGCACTTACT CATTCATTAG GGAGGAAGAT |
| 1551 | CAATGCACCT TCCAACACCG CTCCTTCAGG GCTAATGATT CCTTAGGATT |
| 1601 | TATGCTGCTT CTTGCTCTCA TCCTCCTAGC CACACAGCTT GTCTACCTCA |
| 1651 | AGCTGATATT TTTCGTCCAC GATCGAAGAA AAATGAAGCC AGTCCAGTTT |
| 1701 | GTAGCAGCAG TCAGCCAGAA CTGGACTTTT CATGGTCCTG GAGCCAGTGG |
| 1751 | CCAGGCAGCT GCCAATTGGC TAGCAGGATT TGGAAGGGGT CCCACACCAC |
| 1801 | CCACCTTGCT GGGCATCAGG CAAAATGCAA ACACCACAGG CAGAAGAAGG |
| 1851 | CTATTGGTCT TAGACGAGTT CAAAATGGAG AAAAGAATCA GCAGAATGTT |
| 1901 | CTATATAATG ACTTTTCTGT TTCTAACCTT GTGGGGCCCC TACCTGGTGG |
| 1951 | CCTGTTATTG GAGAGTTTTT GCAAGAGGGC CTGTAGTACC AGGGGGATTT |
| 2001 | CTAACAGCTG CTGTCTGGAT GAGTTTTGCC CAAGCAGGAA TCAATCCTTT |
| 2051 | TGTCTGCATT TTCTCAAACA GGGAGCTGAG GCGCTGTTTC AGCACAACCC |
| 2101 | TTCTTTACTG CAGAAAATCC AGGTTACCAA GGGAACCTTA CTGTGTTATA |
| 2151 | TGAGGGAGCA TCTGTAAATC TTTAGCCTTG TGAAAACTAA CCTTCTCTGC |
| 2201 | TGAGCAATTG TGGCCCATAG CCATATTTTG AGAAGAAATT CAAGAATGGA |
| 2251 | ATCAGCAGTT TTAAGGATTT GGGCAACATT CTGCAGTCTT TGCAAT/AAGTT |
| 2301 | CACCTATAAT CCTATTTTAA ATCTCAGAGT GATCCTGCTG ACTGCCAGCA |
| 2351 | AAGGTTTGTA ATTAAGAAG/AG GACTGAACCA CTGCCCTAAG TTTCTTTATG |
| 2401 | TGGTCAAAAA CTAGATAATG AAAGTAGCAG GTGCTAAGTA TCAGTGCTAA |
| 2451 | ATGCTCTGTA TGTCACTACA TATGAAAAAA CATCAAAAAA CAATTAGCAT |
| 2501 | TGGACATCTT AATAAATTAA GTTGACATGA GGTAAATGTG TTGATAAAAA |
| 2551 | CTAATTTTAG AAGTTTGAAG ACTTTAAAAC ATTTCATACT ACTATTGTTT |
| 2601 | TGCAAAGACT AAAATATTTG GGGACTTAAA GTACTGTAAT CCACTAAAGA |
| 2651 | CGTGCCAATG AATTATTGGA ATATCACACT TTAAAAACCG CCTTGTAAGT |
| 2701 | TCTGGGGAGC ATTCCAAAGC AGTA/GTATTGG TTCCAATTAG AGTTTACTTT |
| 2751 | TTTTGTATTA ATACATTGCT ATTTCTAAAT ACCACTTTCC TCATCTACTA |
| 2801 | GTAAGATTGC TAGCATTGAA CTGTATTATG TGGTTTTTGT TGATTTGGTA |
| 2851 | TAAAGTTTTT CCAATTCATT TATATTTTAC AAATGCTAGA TATTGGTCTG |
| 2901 | GGAGGCAACA TTAATGGTAC CAGCCTGTCA CAACTGAGCA GTTCTAATAA |
| 2951 | TGCAGAATAA ATACATGTTG CCTTAAAGGG TTATCTAGTA TCCTTCATCT |
| 3001 | TATTTAGCAC TGGAGCAAAT AGCCAAGGGA AATCAAATCA GTAACTGGTC |
| 3051 | ATGGTCATGC ATCT |

The sequence set forth above is hereinafter referred to as SEQ ID NO:1

The invention comprises the first description of polynucleotide fragments derived from the sequence of the human G protein coupled receptor Con-202 gene suitable for the diagnosis of schizophrenia or predicting the likelihood of developing schizophrenia. The invention further comprises methods of diagnosis and prediction. One embodiment of the invention encompasses an isolated polynucleotide consisting of between 12 and 200 contiguous nucleotides of SEQ ID NO 1 or its complement including at least one Con-202 polymorphic site selected from the group consisting of the polymorphic sites at positions 184, 1022, 1292, 1400, 2296, 2369 and 2724. This definition and all other definitions below making use of the term between 12 and 200 contiguous nucleotides” is meant to included polynucleotides of each and every integer value between 12 and 200 nucleotides in length.

The invention therefore also encompasses primers or probes comprising one or more single nucleotide polymorphisms which hybridize under stringent conditions to SEQ ID NO:1 or its complement.

The invention provides an isolated polynucleotide consisting of between 12 and 200 contiguous nucleotides of SEQ ID NO 1 in which the nucleotide position 184 is selected from the group of nucleotides G or C

The invention provides an isolated polynucleotide consisting of between 12 and 200 contiguous nucleotides of SEQ ID NO 1 in which the nucleotide position 1022 is selected from the group of nucleotides C or A

The invention provides an isolated polynucleotide consisting of between 12 and 200 contiguous nucleotides of SEQ ID NO 1 in which the nucleotide position 1292 is selected from the group of nucleotides C or T

The invention provides an isolated polynucleotide consisting of between 12 and 200 contiguous nucleotides of SEQ ID NO 1 in which the nucleotide position 1400 is selected from the group of nucleotides C or T

The invention provides an isolated consisting of between 12 and 200 contiguous nucleotides of SEQ ID NO 1 in which the nucleotide position 2296 is selected from the group of nucleotides A or T

The invention provides an isolated polynucleotide consisting of between 12 and 200 contiguous nucleotides of SEQ ID NO 1 in which the nucleotide position 2369 is selected from the group of nucleotides G or A

The invention provides an isolated polynucleotide consisting of between 12 and 200 contiguous nucleotides of SEQ ID NO 1 in which the nucleotide position 2724 is selected from the group of nucleotides A or G.

Complements of these segments are also included. The segments can be DNA or RNA, and can be double- or single-stranded. Some segments are 10-20 or 10-50 bases long. Preferred segments are about 12-200 bases long.

The invention further provides allele-specific oligonucleotides that hybridizes to a sequence shown in (SEQ ID NO: 1), or its complement. These oligonucleotides can be probes or primers.

The invention further provides a method for classifying a nucleic acid molecule encoding Con-202 or a fragment thereof from an individual comprising obtaining said nucleic acid from said individual; and determining a nucleotide from said nucleic acid which corresponds to the nucleotide occupying at least one polymorphic site selected from the group consisting of the polymorphic sites at positions 184, 1022, 1292, 1400, 2296, 2369 and 2724

The present invention therefore further provides a method of diagnosing schizophrenia or determining a predisposition to schizophrenia by determining the presence or absence of a Con-202 haplotype in a patient by obtaining material from a patient comprising nucleic acid including one or more of the nucleotides at position, 184, 1022, 1292, 1400, 2296, 2369 and 2724 of SEQ ID NO:1 and determining the Con-202 haplotype.

### BRIEF DESCRIPTION OF THE FIGURES

FIG. 1 Organization of the Con-202 Gene with the location of polymorphism noted

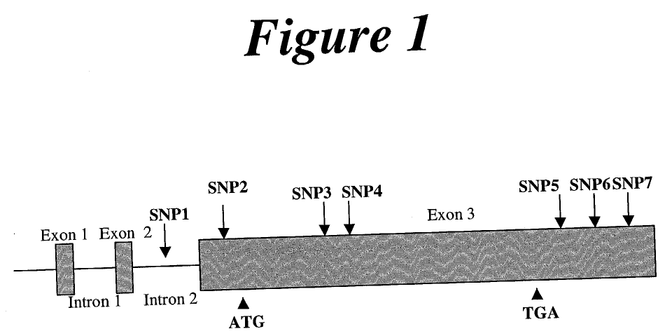

### BRIEF DESCRIPTION OF THE SEQUENCE LISTING

SEQ ID NO:1 The native DNA sequence of Con-202 with variation noted at positions 184, 1022, 1292, 1400, 2296, 2369 and 2724.

SEQ ID NO:2 PCR Primer—Example 1

SEQ ID NO:3 PCR Primer—Example 1

SEQ ID NO:4 PCR Primer—Example 1

SEQ ID NO:5 PCR Primer—Example 1

SEQ ID NO:6 TaqMan Probe—Table 3

SEQ ID NO:7 TaqMan Probe—Table 3

SEQ ID NO:8 TaqMan Probe—Table 3

SEQ ID NO:9 TaqMan Probe—Table 3

SEQ ID NO:10 TaqMan Probe—Table 3

SEQ ID NO:11 TaqMan Probe—Table 3

SEQ ID NO:12 TaqMan Probe—Table 3

SEQ ID NO:13 TaqMan Probe—Table 3

SEQ ID NO:14 TaqMan Probe—Table 3

SEQ ID NO:15 TaqMan Probe—Table 3

SEQ ID NO:16 PCR Primer—Table 3

SEQ ID NO:17 PCR Primer—Table 3

SEQ ID NO:18 PCR Primer—Table 3

SEQ ID NO:19 PCR Primer—Table 3

SEQ ID NO:20 PCR Primer—Table 3

SEQ ID NO:21 PCR Primer—Table 3

SEQ ID NO:22 PCR Primer—Table 3

SEQ ID NO:23 PCR Primer—Table 3

SEQ ID NO:24 PCR Primer—Table 3

SEQ ID NO:25 PCR Primer—Table 3

### DETAILED DESCRIPTION OF THE INVENTION

Definitions

The term “allele” is used herein to refer to variants of a nucleotide sequence.

An “agent acting on schizophrenia” includes any drug or compound known in the art that addresses, reduces or alleviates one or more symptoms of schizophrenia. “Agents acting on a schizophrenia” includes any drug or a compound modulating the activity or concentration of an enzyme or regulatory molecule involved in a schizophrenia that is known in the art. Agents acting on schizophrenia include but are not limited to Thorazine, Mellaril, Modecate, Prolixin, Navane, Stelazine and Haldol, risperidone (Risperdal), clozapine (Clozaril), olanzapine (Zyprexa) and quetiapine (Seroquel).

The term “response to an agent acting on schizophrenia” refer to drug efficacy, including but not limited to the ability to metabolize a compound, the ability to convert a pro-drug to an active drug, and to the pharmacokinetics (absorption, distribution, elimination) and the pharmacodynamics (receptor-related) of a drug in an individual. The terms “side effects to an agent acting on schizophrenia” refer to adverse effects of therapy resulting from extensions of the principal pharmacological action of the drug or to idiosyncratic adverse reactions resulting from an interaction of the drug with unique host factors. “Side effects to an agent acting on schizophrenia” include, but are not limited to autonomic side effects such as orthostatic hypotension, blurred vision, dry mouth, nasal congestion and constipation. “Side effects to an agent acting on schizophrenia” also include anxiety, sleep disturbances, sexual dysfunction, gastrointestinal disturbances, nausea, diarrhea, orthostasis, dizziness, sedation, hypertension, shock, akinesia (slowed movement), akathisia (restless limbs), and tardive dyskinesia (permanent, irreversible movement disorders.)

The terms “complementary” or “complement thereof” are used herein to refer to the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

The term “genotype” as used herein refers the identity of the alleles present in an individual or a sample. In the context of the present invention a genotype preferably refers to the description of the polymorphic alleles present in an individual or a sample. The term “genotyping” a sample or an individual for a polymorphic marker consists of determining the specific allele or the specific nucleotide carried by an individual at a polymorphic marker.

The term “heterozygosity rate” is used herein to refer to the incidence of individuals in a population, which are heterozygous at a particular allele. In a polymorphic system the heterozygosity rate is on average equal to 2Pa(1-Pa), where Pa is the frequency of the least common allele. In order to be useful in genetic studies a genetic marker should have an adequate level of heterozygosity to allow a reasonable probability that a randomly selected person will be heterozygous.

The term “mutation” as used herein refers to a difference in DNA sequence between or among different genomes or individuals which has a frequency below 1%.

The term “haplotype” refers to the actual combination of alleles on one chromosome. In the context of the present invention a haplotype preferably refers to a combination of polymorphisms found in a given individual and which may be associated with a phenotype.

The term “polymorphism” as used herein refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. Polymorphism refers to the occurrence of two or more genetically determined alternative sequences or alleles in a population. Preferred polymorphisms have at least two alleles, each occurring at frequency of greater than 1%, and more preferably greater than 10% or 20% of a selected population. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wild type form. Diploid organisms may be homozygous or heterozygous for allelic forms. A biallelic polymorphism has two forms. A triallelic polymorphism has three forms.

A “single nucleotide polymorphism” (SNP) is a single base pair change. A single nucleotide polymorphism occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations).

A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele. It should be noted that a single nucleotide change could result in the destruction or creation of a restriction site. Therefore it is possible that a single nucleotide polymorphism might also present itself as a restriction fragment length polymorphism.

Single nucleotide polymorphisms (SNPs) can be used in the same manner as RFLPs, and VNTRs but offer several advantages. Single nucleotide polymorphisms occur with greater frequency and are spaced more uniformly throughout the genome than other forms of polymorphism. (SNPs) occur at a frequency of roughly 1/1000 base pairs, and are distinguished from rare variations or mutations by a requirement for the least abundant allele to have a frequency of 1% or more (Brookes, 1999).

Examples of SNP Include:

1. Non-synonymous coding region changes which substitute one amino acid for another in the protein product encoded by the gene,

2. Synonymous changes which do alter amino acid coding sequence due to degeneracy of the genetic code,

3. Changes in promoter, enhancer or other genetic control element sequence which may or may not alter transcription of the gene,

4. Changes in untranslated regions of the mRNA, particularly at the 5'end which may alter the efficiency of ribosomal binding, initiation or translation, or at the 3'end which may alter mRNA stability, and

5. Changes within intronic regions which may alter the splicing of the transcript or the function of other genetic regulatory elements.

The terms “biallelic polymorphism” and “biallelic marker” are used interchangeably herein to refer to a polymorphism having two alleles at a fairly high frequency in the population, preferably a single nucleotide polymorphism. A “biallelic marker allele” refers to the nucleotide variants present at a biallelic marker site. Typically the frequency of the less common allele of the biallelic markers of the present invention has been validated to be greater than 1%, preferably the frequency is greater than 10%, more preferably the frequency is at least 20% (i.e. heterozygosity rate of at least 0.32), even more preferably the frequency is at least 30% (i.e. heterozygosity rate of at least 0.42). A biallelic marker wherein the frequency of the less common allele is 30% or more is termed a “high quality biallelic marker.

The term “Con-202 polymorphism” or “Con-202 polymorphic site” is used herein to mean a polymorphism or polymorphic site within the gene for Con-202 disclosed herein. This term would encompass polymorphisms at polymorphic sites within the Con-202 coding sequence, intronic regions and flanking regions. A “Con-202 polymorphism” or need not change an amino acid in the Con-202 protein product in order to have utility. The term Con-202 polymporphism encompasses single nucleotide polymorphisms, biallelic and otherwise and are the polymorphisms described in Table 1 of this disclosure. A Con-202 single nucleotide polymorphism is a polymorphism which reflects variation at a single nucleotide. The term “at least one Con-202 polymorphic site” means at least one polymorphic site within the Con-202 gene selected from those detailed in Table 1 of this disclosure.

As used interchangeably herein, the term “oligonucleotides”, and “polynucleotides” include RNA, DNA, or RNA/DNA hybrid sequences of more than one nucleotide in either single chain or duplex form. The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. Although the term “nucleotide” is also used herein to encompass “modified nucleotides” which comprise at least one modifications (a) an alternative linking group, (b) an analogous form of purine, (c) an analogous form of pyrimidine, or (d) an analogous sugar, for examples of analogous linking groups, purine, pyrimidines, and sugars see for example PCT publication No. WO 95/04064. However, the polynucleotides of the invention are preferably comprised of greater than 50% conventional deoxyribose nucleotides, and most preferably greater than 90% conventional deoxyribose nucleotides The polynucleotide sequences of the invention may be prepared by any known method, including synthetic, recombinant, ex vivo generation, or a combination thereof, as well as utilizing any purification methods known in the art.

The location of nucleotides in a polynucleotide with respect to the center of the polynucleotide are described herein in the following manner. When a polynucleotide has an odd number of nucleotides, the nucleotide at an equal distance from the 3′ and 5′ ends of the polynucleotide is considered to be “at the center” of the polynucleotide, and any nucleotide immediately adjacent to the nucleotide at the center, or the nucleotide at the center itself is considered to be “within 1 nucleotide of the center.” With an odd number of nucleotides in a polynucleotide any of the five nucleotides positions in the middle of the polynucleotide would be considered to be within 2 nucleotides of the center, and so on. When a polynucleotide has an even number of nucleotides, there would be a bond and not a nucleotide at the center of the polynucleotide. Thus, either of the two central nucleotides would be considered to be “within 1 nucleotide of the center” and any of the four nucleotides in the middle of the polynucleotide would be considered to be “within 2 nucleotides of the center”, and so on. For polymorphisms which involve the substitution, insertion or deletion of 1 or more nucleotides, the polymorphism, allele or biallelic marker is “at the center” of a polynucleotide if the difference between the distance from 3′ the substituted, inserted, or deleted polynucleotides of the polymorphism and the 3′ end of the polynucleotide, and the distance from the substituted, inserted, or deleted polynucleotides of the polymorphism and the 5′ end of the polynucleotide is zero or one nucleotide. If this difference is 0 to 3, then the polymorphism is considered to be “within 1 nucleotide of the center.” If the difference is 0 to 5, the polymorphism is considered to be “within 2 nucleotides of the center.” If the difference is 0 to 7, the polymorphism is considered to be “within 3 nucleotides of the center,” and so on. For polymorphisms which involve the substitution, insertion or deletion of 1 or more nucleotides, the polymorphism, allele or biallelic marker is “at the center” of a polynucleotide if the difference between the distance from the substituted, inserted, or deleted polynucleotides of the polymorphism and the 3′ end of the polynucleotide, and the distance from the substituted, inserted, or deleted polynucleotides of the polymorphism and the 5′ end of the polynucleotide is zero or one nucleotide. If this difference is 0 to 3, then the polymorphism is considered to be “within 1 nucleotide of the center.” If the difference is 0 to 5, the polymorphism is considered to be “within 2 nucleotides of the center.” If the difference is 0 to 7, the polymorphism is considered to be “within 3 nucleotides of the center,” and so on.

The location of nucleotides in a polynucleotide with respect to the end of the polynucleotide are described herein in the following manner. A nucleotide is “at the end” of a polynucleotide if it is at either the 5′ or 3′ end of the polynucleotide.

The term “upstream” is used herein to refer to a location which, is toward the 5′ end of the polynucleotide from a specific reference point. The terms “base paired” and “Watson & Crick base paired” are used interchangeably herein to refer to nucleotides which can be hydrogen bonded to one another be virtue of their sequence identities in a manner like that found in double-helical DNA with thymine or uracil residues linked to adenine residues by two hydrogen bonds and cytosine and guanine residues linked by three hydrogen bonds (See Stryer, L., Biochemistry, 4^{th} edition, 1995).

The term “purified” is used herein to describe a polynucleotide or polynucleotide vector of the invention which has been separated from other compounds including, but not limited to other nucleic acids, carbohydrates, lipids and proteins (such as the enzymes used in the synthesis of the polynucleotide), or the separation of covalently closed polynucleotides from linear polynucleotides. A polynucleotide is substantially pure when at least about 50%, preferably 60 to 75% of a sample exhibits a single polynucleotide sequence and conformation (linear versus covalently closed). A substantially pure polynucleotide typically comprises about 50%, preferably 60 to 90% weight/weight of a nucleic acid sample, more usually about 95%, and preferably is over about 99% pure. Polynucleotide purity or homogeneity may be indicated by a number of means well known in the art, such as agarose or polyacrylamide gel electrophoresis of a sample, followed by visualizing a single polynucleotide band upon staining the gel. For certain purposes higher resolution can be provided by using HPLC or other means well known in the art.

The term primer refers to a single-stranded oligonucleotide capable of acting as a point of initiation of template-directed DNA synthesis under appropriate conditions (i.e., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer but typically ranges from 15 to 30 nucleotides. Short primer molecules generally require cooler temperatures to form sufficiently stable hybrid complexes with the template. A primer need not reflect the exact sequence of the template but must be sufficiently complementary to hybridize with a template. The term primer site refers to the area of the target DNA to which a primer hybridizes. The term primer pair means a set of primers including a 5′ upstream primer that hybridizes with the 5′ end of the DNA sequence to be amplified and a 3′, downstream primer that hybridizes with the complement of the 3′ end of the sequence to be amplified.

The term “probe” or “hybridization probe” denotes a defined nucleic acid segment (or nucleotide analog segment, e.g., polynucleotide as defined herein) which can be used to identify a specific polynucleotide sequence present in samples, said nucleic acid segment comprising a nucleotide sequence complementary of the specific polynucleotide sequence to be identified by hybridization. “Probes” or “hybridization probes' are nucleic acids capable of binding in a base-specific manner to a complementary strand of nucleic acid. Such probes include peptide nucleic acids, as described in Nielsen et al., Science 254, 1497-1500 (1991). Hybridizations are usually performed under “stringent conditions”, for example, at a salt concentration of no more than 1M and a temperature of at least 25° C. For example, conditions of 5×SSPE (750 mM NaCl, 50 mM NaPhosphate, 5 mM EDTA, pH 7.4) and a temperature of 25°-30° C. are suitable for allele-specific probe hybridizations. Although this particular buffer composition is offered as an example, one skilled in the art, could easily substitute other compositions of equal suitability.

The term “sequencing” as used herein, means a process for determining the order of nucleotides in a nucleic acid. A variety of methods for sequencing nucleic acids are well known in the art. Such sequencing methods include the Sanger method of dideoxy-mediated chain termination as described, for example, in Sanger et al., Proc. Natl. Acad. Sci. 74:5463 (1977), which is incorporated herein by reference (see, also, “DNA Sequencing” in Sambrook et al. (eds.), Molecular Cloning: A Laboratory Manual (Second Edition), Plainview, N.Y.: Cold Spring Harbor Laboratory Press (1989), which is incorporated herein by reference). A variety of polymerases including the Klenow fragment of E. coli DNA polymerase I; Sequenase TM (T7 DNA polymerase); Taq DNA polymerase and Amplitaq can be used in enzymatic sequencing methods. Well known sequencing methods also include Maxam-Gilbert chemical degradation of DNA (see Maxam and Gilbert, Methods Enzymol. 65:499 (1980), which is incorporated herein by reference, and “DNA Sequencing” in Sambrook et al., supra, 1989). Once skilled in the art recognizes that sequencing is now often performed with the aid of automated methods.

The term “schizophrenia” refers has its conventional meaning, e.g., a mental disorder characterized by the constellation of symptoms described in the DSM-III-R.

The terms “trait” and “phenotype” are used interchangeably herein and refer to any visible, detectable or otherwise measurable property of an organism such as symptoms of, or susceptibility to a disease for example. Typically the terms “trait” or “phenotype” are used herein to refer to symptoms of, or susceptibility to schizophrenia; or to refer to an individual's response to an agent acting on schizophrenia; or to refer to symptoms of, or susceptibility to side effects to an agent acting on schizophrenia.

Polymorphisms of the Invention

The nucleotide and amino acid sequence of the Con-202 cDNA has been disclosed previously in U.S. patent application Ser. No. 09/427, 653 filed on Oct. 27, 1999 and U.S. patent application Ser. No. 09/698,419 filed Oct. 27, 2000 both of which are herein incorporated by reference

In situ hybridization analysis shows the presence of Con-202 mRNA in cortical regions, lateral olfactory nucleus (Lat. olf. nu.), hippocampus, subthalamic nucleus, and at a low level in the substantia nigra-pars compacta. The regions where Con-202 are expressed are within the limbic and neuroendocrine circuitry of the brain.

The chromosomal location of the gene encoding Con-202 was determined using the Stanford G3 Radiation Hybrid Panel (Research Genetics, Inc., Huntsville, Ala.). This panel contains 83 radiation hybrid clones of the entire human genome created by the Stanford Human Genome Center. PCR primers were designed from the sequence in SEQ ID NO:1 to determine which lanes gave a PCR product. Lanes were scored for the presence or absence of the expected PCR product and the results submitted to the Stanford Human Genome Center via e-mail for analysis. The analysis of the Con-202 places it on chromosome 7, most nearly linked to Stanford marker SHGC-12021 with a LOD score of 10.36. This marker lies at position 7q31. One study looking at schizophrenia in the Finnish population found a region on chromosome 7q22 that showed linkage with schizophrenia (Ekelund 2000). The maximum lod score of 3.53 was between markers D7S501 and D7S523. While the strongest evidence for linkage is between markers D7S501 and D7S523, there is evidence of linkage of schizophrenia to chromosome 7 in markers spanning approximately 12 centimorgan (cM). The marker SHGC-12021, where Con-202 is located, is approximately one cM from the marker D7S523, placing Con-202 within the 12 cM span. We have identified 7 single nucleotide polymorphisms (SNPs) within the Con-202 gene sequence.

TABLE 1
Con-202 SNPs

| SNP | Location (base) | Common/Rare Allele |
|---|---|---|
| S1 | 184 | G/C |
| S2 | 1022 | C/A |
| S3 | 1292 | C/T |
| S4 | 1400 | C/T |
| S5 | 2296 | T/A |
| S6 | 2369 | G/A |
| S7 | 2724 | A/G |

As noted above, the Con-202 single nucleotide polymorphism at position 184 of SEQ ID NO:1 is designated SI, the Con-202 single nucleotide polymorphism at position 1022 of SEQ ID NO:1 is designated S2, the Con-202 single nucleotide polymorphism at position 1292 of SEQ ID NO:1 is designated S3, the Con-202 single nucleotide polymorphism at position 1400 of SEQ ID NO:1 is designated S4, the Con-202 single nucleotide polymorphism at position 2296 of SEQ ID NO:1 is designated S5, the Con-202 single nucleotide polymorphism at position 2369 of SEQ ID NO:1 is designated S6, and the Con-202 single nucleotide polymorphism at position 2724 of SEQ ID NO:1 is designated S7.

There are two distinct types of analysis depending whether a polymorphism in question has already been characterized. The first type of analysis is sometimes referred to as de novo identification. The second type of analysis is determining which form(s) of an identified polymorphism are present in individuals under test. The first type of analysis compares target sequences in different individuals to identify points of variation, i.e., polymorphic sites. By analyzing a groups of individuals representing the greatest ethnic diversity among humans and greatest breed and species variety in plants and animals, patterns characteristic of the most common alleles/haplotypes of the locus can be identified, and the frequencies of such populations in the population determined. Additional allelic frequencies can be determined for subpopulations characterized by criteria such as geography, race, or gender. An example describing the de-novo identification of the polymorphisms of the invention is described below.

### EXAMPLE 1

### De-Novo Identification of Polymorphisms of the Invention

Materials and Methods

DNA Samples

DNA samples were obtained from anonymous blood samples from asymptomatic individuals. DNA was prepared using the QiaAmp DNA blood mini kit (Qiagen). The samples are referred to as the Population Control Western Michigan samples and labeled CON01.

PCR Amplification of Con-202

The fragment spanning nucleotide 1-1271 of SEQ ID NO: 1) was generated by PCR in a 50 μl reaction containing 32 μl of H₂O, 5 μl of 10×TT buffer (140 mM Ammonium Sulfate, 0.1% gelatin, 0.6 M Tris-tricine pH 8.4), 5 μl of 15 mM MgSO₄, 2 μl of 10 mM dNTP, 5 μl of CON01 DNA (5 μg/μl), 0.3 μl LW1776 (1 μg/μl)(GAGGAAGCCGATTTCCCTGG—SEQ ID NO:2), 0.3 μl LW1478 (1 μg/μl)(CACAAATGGGAAACAAATTGC—SEQ ID NO:3), 0.4 μl Expand High Fidelity enzyme mix (3.5 U/μl)(Roche). The PCR reaction was incubated in a GeneAmp 9600 PCR thermocycler (Perkin Elmer Applied Biosystems). The cycling program was as follows: 1 cycle of 94° C. for 2 min then 10 cycles at [(94° C. for 15 sec)→(68° C. for 2 min decreasing 1° C. each cycle)→(72° C. for 1.3 min)], followed by 50 cycles at [(94° C. for 15 sec)→(58° C. for 30 sec)→(72° C. for 1.3 min)], then 72° C. for 7 min.

The fragment spanning nucleotide 1037-3064 of SEQ ID NO: 1 was generated by PCR in a 50 μl reaction containing 37 μl of H₂O, 5 μl of 10×TT buffer, 5 μl of 15 mM MgSO₄, 2 μl of 10 mM dNTP, 0.3 μl LW1482 (1 μg/μl) (AGCTATGGCGAACTATAGCCATGCAGC—SEQ ID NO:4), 0.3 μl LW1777 (1 μg/μl) (AGATGCATGACCATGACCAG—SEQ ID NO:5), 0.4 μl Expand High Fidelity enzyme mix (3.5 U/1)(Roche) and 25 ng of dried down DNA from CON01. The PCR reaction was incubated in a GeneAmp 9600 PCR thermocycler (Perkin Elmer Applied Biosystems). The cycling program was as follows: 1 cycle of 94° C. for 2 min then 10 cycles at [(94° C. for 15 sec)→(66° C. for 2 min decreasing 1° C. each cycle)→(72° C. for 2 min)], followed by 50 cycles at [(94° C. for 15 sec)→(56° C. for 30 sec)→(72° C. for 2 min)], then 72° C. for 7 min.

The PCR products were purified using MultiScreen-PCR Filter Plates (Millipore). The PCR reaction was loaded onto the plate and the plate was placed on top of the MultiScreen manifold (Millipore) and a vacuum of 24 inches Hg was applied for 5-10 minutes. The plate was removed from the manifold and 50 μl of H₂O was added to each well. The plate was placed on a plate mixer and shook vigorously for 5 minutes. The purified PCR product was recovered from each well and placed into a new 96 well reaction plate.

DNA Sequencing

The PCR fragments were sequenced directly using an ABI377 fluorescence-based sequencer (Perkin Elmer/Applied Biosystems Division, PE/ABD, Foster City, Calif.) and the ABI BigDye™ Terminator Cycle Sequencing Ready Reaction kit with Taq FSTM polymerase. Each cycle-sequencing reaction contained 9.6 μl of H₂O, 8.4 μl of BigDye Terminator mix (8 μl of Big Dye Terminator and 0.4 μl of DMSO), 1 μl DNA (˜0.5 μg), and 1 μl primer (25 ng/μl) and was performed in a Perkin-Elmer 9600. Cycle-sequencing was performed using an initial denaturation at 98° C. for 1 min, followed by 50 cycles: 96° C. for 30 sec, annealing at 50° C. for 30 sec, and extension at 60° C. for 4 min Extension products were purified using AGTC® gel filtration block (Edge BiosSystems, Gaithersburg, Md.). Each reaction product was loaded by pipette onto the column, which was then centrifuged in a swinging bucket centrifuge (Sorvall model RT6000B tabletop centrifuge) at 750×g for 2 min at room temperature. Column-purified samples were dried under vacuum for about 60 min and then dissolved in 2 μl of a DNA loading solution (83% deionized formamide, 8.3 mM EDTA, and 1.6 mg/ml Blue Dextran). The samples were then heated to 90° C. for 2.3 min and 0.75 μl of each sample was loaded into the gel sample wells for sequence analysis by the ABI377 sequencer. The sequence chromatograms were analyzed using the computer program POLYPHRED. Nickerson, D. A. (1997) Nucleic Acids Research, 25(14), pp. 2745-2751.

Results

A plate containing the DNA from 72 individuals, which we refer to as the Population Control Western Michigan samples (labeled CON01), was amplified using primers described above. The PCR products were purified and sequenced. The chromotograms were analyzed with the computer program POLYPHRED, which compares the sequence of the 72 individuals and indicates differences in the sequence. A total of seven SNPs were identified. A summary of the results is shown in Table 2 below.

TABLE 2

| Ref- | | | | Common | | Rare | Per- |
|---|---|---|---|---|---|---|---|
| erence | Base | DNA | | homo- | Hetero- | homo- | cent |
| se- | SNP | region | Effect | zygotes | zygotes | zygotes | rare |
| quence | found | found | of SNP | found | found | found | allele |
| S1 | 184 | Intron 2 | | 53 GG | 18 C/G | 1 CC | 13.9 |
| S2 | 1022 | 5' UTR | | 45 CC | 22 C/A | 4 AA | 21.1 |
| S3 | 1292 | coding | silent | 53 CC | 15 C/T | 0 | 10.9 |
| S4 | 1400 | coding | silent | 54 CC | 15 C/T | 0 | 10.9 |
| S5 | 2296 | 3'UTR | | 56 TT | 10 A/T | 0 | 7.5 |
| S6 | 2369 | 3'UTR | | 59 GG | 4 G/A | 0 | 3.2 |
| S7 | 2724 | 3'UTR | | 24 AA | 23 G/A | 9 GG | 37 |

One SNP is in intron 2, one in the 5′ untranslated region (19 base pairs 5′ of the initiation methione), two in the coding region and three in the 3′ untranslated region. The locations of the SNPs are at nucleotide 184, 1022, 1292, 1400, 2296, 2369, and 2724, relative to the sequence in SEQ ID NO:1. The frequency of the rare allele for each SNP is 13.9, 21.1, 10.9, 10.9, 7.5, 3.2 and 37% respectively. Neither of the SNPs in the coding region changes an amino acid but a comparison of the 72 individuals suggust that the two SNPs segregate together but without knowing the haplotype of the heterozyotes its possible that the two SNPs are not linked. The genomic clone we isolated for Con-202 contains the rare allele for the two SNPs found in the coding region.

Association Studies

Once a polymorphism is identified, as noted above, it becomes desireable to determine which form(s) of an identified polymorphism are present in individuals under test. This becomes important in characterizing the polymorphisms further as to their possible association with a disease state. Once such an association is determined the same methods may, of course be used for diagnostic and predictive purposes. In determining the identity of a particular nucleotide position there are a variety of suitable procedures, which are discussed in turn.

Analysis of Polymorphisms

A. Preparation of Samples

Polymorphisms are detected in a target nucleic acid from an individual being analyzed. For assay of genomic DNA, virtually any biological sample (other than pure red blood cells) is suitable. For example, convenient tissue samples include whole blood, semen, saliva, tears, urine, fecal material, sweat, buccal, skin and hair. For assay of cDNA or mRNA, the tissue sample must be obtained from an organ in which the target nucleic acid is expressed.

Many of the methods described below require amplification of DNA from target samples. This can be accomplished by PCR. See generally PCR Technology: Principles and Applications for DNA Amplification (ed. H. A. Erlich, Freeman Press, N.Y., N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202 (each of which is incorporated by reference for all purposes).

Other suitable amplification methods include the ligase chain reaction (LCR) (see Wu and Wallace, Genomics 4, 560 (1989), Landegren et al., Science 241, 1077 (1988), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

B. Detection of Polymorphisms in Target DNA

1. Allele-Specific Probes

The design and use of allele-specific probes for analyzing polymorphisms is described by e.g., Saiki et al., Nature 324, 163-166 (1986); Dattagupta, EP 235,726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Some probes are designed to hybridize to a segment of target DNA such that the polymorphic site aligns with a central position (e.g., in a 15 mer at the 7 position; in a 16 mer, at either the 8 or 9 position) of the probe. This design of probe achieves good discrimination in hybridization between different allelic forms.

These probes are characterized in that they preferably comprise between 8 and 50 nucleotides, and in that they are sufficiently complementary to a sequence comprising a polymorphic marker of the present invention to hybridize thereto and preferably sufficiently specific to be able to discriminate the targeted sequence for only one nucleotide variation. The GC content in the probes of the invention usually ranges between 10 and 75%, preferably between 35 and 60%, and more preferably between 40 and 55%. The length of these probes can range from 10, 15, 20, or 30 to at least 100 nucleotides, preferably from 10 to 50, more preferably from 18 to 35 nucleotides. A particularly preferred probe is 25 nucleotides; in length. Preferably the polymorphic marker is within 4 nucleotides of the center of the polynucleotide probe. In particularly preferred probes the polymorphic marker is at the center of said polynucleotide. Shorter probes may lack specificity for a target nucleic acid sequence and generally require cooler temperatures to form sufficiently stable hybrid complexes with the template. Longer probes are expensive to produce and can sometimes self-hybridize to form hairpin structures. Methods for the synthesis of oligonucleotide probes have been described above and can be applied to the probes of the present invention.

Preferably the probes of the present invention are labeled or immobilized on a solid support. Labels and solid supports are well known in the art. Detection probes are generally nucleic acid sequences or uncharged nucleic acid analogs such as, for example peptide nucleic acids which are disclosed in International Patent Application WO 92/20702, morpholino analogs which are described in U.S. Pat. Nos. 5,185,444; 5,034,506 and 5,142,047. The probe may have to be rendered “non-extendable” in that additional dNTPs cannot be added to the probe. In and of themselves analogs usually are non-extendable and nucleic acid probes can be rendered non-extendable by modifying the 3′ end of the probe such that the hydroxyl group is no longer capable of participating in elongation. For example, the 3′ end of the probe can be functionalized with the capture or detection label to thereby consume or otherwise block the hydroxyl group. Alternatively, the 3′ hydroxyl group simply can be cleaved, replaced or modified,

The probes of the present invention are useful for a number of purposes. They can be used in Southern hybridization to genomic DNA or Northern hybridization to mRNA. The probes can also be used to detect PCR amplification products. By assaying the hybridization to an allele. specific probe, one can detect the presence or absence of a biallelic marker allele in a given sample.

High-Throughput parallel hybridizations in array format are specifically encompassed within “hybridization assays” and are described below.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

2. Allele-Specific Primers

An allele-specific primer hybridizes to a site on target DNA overlapping a polymorphism and only primes amplification of an allelic form to which the primer exhibits perfect complementarily. See Gibbs, Nucleic Acid Res. 17, 2427-2448 (1989). This primer is used in conjunction with a second primer which hybridizes at a distal site. Amplification proceeds from the two primers leading to a detectable product signifying the particular allelic form is present. A control is usually performed with a second pair of primers, one of which shows a single base mismatch at the polymorphic site and the other of which exhibits perfect complementarily to a distal site. The single-base mismatch prevents amplification and no detectable product is formed. The method works best when the mismatch is included in the 3′-most position of the oligonucleotide aligned with the polymorphism because this position is most destabilizing to elongation from the primer. See, e.g., WO 93/22456. The invention of course, contemplates such primers with distal mismatches as well as primers which because of chosen conditions form unstable base pairing and thus prime inefficiently.

3. Direct-Sequencing

The direct analysis of the sequence of polymorphisms of the present invention can be accomplished using either the dideoxy chain termination method or the Maxam Gilbert method (see Sambrook et al., Molecular Cloning, A Laboratory Manual (2nd Ed., CSHP, New York 1989); Zyskind et al., Recombinant DNA Laboratory Manual, (Acad. Press, 1988). It should be recognized that the field of DNA sequencing has advanced considerably in the past several years and that the invention contemplates such advances. Most notably, within the past decade there has been increasing reliance on automated DNA sequence analysis.

4. Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W. H. Freeman and Co, New York, 1992), Chapter 7.

5. Single-Strand Conformation Polymorphism Analysis

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence difference between alleles of target sequences.

Other modifications of the methods above exist, including allele-specific hybridization on filters, allele-specific PCR, PCR plus restriction enzyme digest (RFLP-PCR), denaturing capillary electrophoresis, primer extension and time-of-flight mass spectrometry, and the 5′ nuclease (Taq-Man™) assay.

The Taq-Man assay takes advantage of the 5′ nuclease activity of Taq DNA polymerase to digest a DNA probe annealed specifically to the accumulating amplification product. Taq-Man probes are labeled with a donor-acceptor dye pair that interacts via fluorescence energy transfer. Cleavage of the Taq-Man probe by the advancing polymerase during amplification dissociates the donor dye from the quenching acceptor dye, greatly increasing the donor fluorescence. All reagents necessary to detect two allelic variants can be assembled at the beginning of the reaction and the results are monitored in real time (see Livak et al., Nature Genetics, 9:341-342, 1995). In an alternative homogeneous hybridization-based procedure, molecular beacons are used for allele discriminations. Molecular beacons are hairpin-shaped oligonucleotide probes that report the presence of specific nucleic acids in homogeneous solutions. When they bind to their targets they undergo a conformational reorganization that restores the fluorescence of an internally quenched fluorophore (Tyagi et al., Nature Biotechnology, 16:49-531 1998).

Preferred techniques for SNP genotyping should allow large scale, automated analysis which do not require extensive optimization for each SNP analyzed. Examples of the later are DASH (Dynamic Allele-Specific hybridization) which is amenable to formatting in microtiter plates (Hybaid) and “single-stringency” DNA-chip hybridization (Affymetrix)” It should be recognized of course, that this list is not inclusive.

High-Throughput parallel hybridizations in array format are specifically encompassed by the invention and are described below.

Hybridization assays based on oligonucleotide arrays rely on the differences in hybridization stability of short oligonucleotides to perfectly matched and mismatched target sequence variants. Efficient access to polymorphism information is obtained through a basic structure comprising high-density arrays of oligonucleotide probes attached to a solid support (the chip) at selected positions. Each DNA chip can contain thousands to millions of individual synthetic DNA probes arranged in a grid-like pattern and miniaturized to the size of a dime.

The chip technology has already been applied with success in numerous cases. For example, the screening of mutations has been undertaken in the BRCA I gene, in S. cerevisiae mutant strains, and in the protease gene of HIV-I virus (Hacia et al., Nature Genetics, 14(4):441-447, 1996; Shoemaker et al., Nature Genetics, 14(4):450-456, 1996 Kozal et al., Nature Medicine, 2:753-759, 1996). Chips of various formats for use in detecting biallelic polymorphisms can be produced on a customized basis by Affymetrix (GeneChip™), Hyseq (HyChip and HyGnostics), and Protogene Laboratories.

In general, these methods employ arrays of oligonucleotide probes that are complementary to target nucleic acid sequence segments from an individual which, target sequences include a polymorphic marker. EP785280 describes a tiling strategy for the detection of single nucleotide polymorphisms. Briefly, arrays may generally be “tiled” for a large number of specific polymorphisms. By “tiling” is generally meant the synthesis of a defined set of oligonucleotide probes which is made up of a sequence complementary to the target sequence of interest, as well as preselected variations of that sequence, e.g., substitution of one or more given positions with one or more members of the basis set of monomers, i.e. nucleotides. Tiling strategies are further described in PCT application No. WO 95/11995. In a particular aspect, arrays are tiled for a number of specific, identified biallelic marker sequences. In particular the array is tiled to include a number of detection blocks, each detection block being specific for a specific biallelic marker or a set of biallelic markers. For example, a detection block may be tiled to include a number of probes, which span the sequence segment that includes a specific polymorphism. To ensure probes that are complementary to each allele, the probes are synthesized in pairs differing at the biallelic marker. In addition to the probes differing at the polymorphic base, monosubstituted probes are also generally tiled within the detection block. These monosubstituted probes have bases at and up to a certain number of bases in either direction from the polymorphism, substituted with the remaining nucleotides (selected from A, T, G, C and U). Typically the probes in a tiled detection block will include substitutions of the sequence positions up to and including those that are 5 bases away from the biallelic marker. The monosubstituted probes provide internal controls for the tiled array, to distinguish actual hybridization from artefactual crosshybridization. Upon completion of hybridization with the target sequence and washing of the array, the array is scanned to determine the position on the array to which the target sequence hybridizes. The hybridization data from the scanned array is then analyzed to identify which allele or alleles of the biallelic marker are present in the sample. Hybridization and scanning may be carried out as described in PCT application No. WO 92/10092 and WO 95/11995 and U.S. Pat. No. 5,424,186.

Thus, in some embodiments, the chips may comprise an array of nucleic acid sequences of fragments of about 15 nucleotides in length. In further embodiments, the chip may comprise an array including at least one of the sequences selected from the group consisting of an isolated polynucleotide comprising between 6-800 contiguous nucleotides of SEQ ID No. 1 and the sequences complementary thereto, or a fragment thereof at least about 8 consecutive nucleotides, preferably 10, 15, 20, more preferably 25, 30, 40, 47, or 50 consecutive nucleotides, including at least one polymorphic site. In some embodiments, the chip may comprise an array of at least 2, 3, 4, 5, 6, 7, 8 or more of these polynucleotides of the invention. Solid supports and polynucleotides of the present invention attached to solid supports are further described in 1.

Fluorescent Allele-Specific PCR (FAS-PCR) uses allele specific primers which differ by a single 3′ nucleotide which is an exact match to the allele to be detected (Howard et al. 1999). Thus, two primers designed to match exactly each allele of a biallelic SNP are used with a single, common, reverse primer to detect each of the allele specific primers. This uses to advantage the observation that if the 3′ nucleotide of the PCR amplification primer does not match exactly, then amplification will not be successful. Typically, each allele specific primer is tagged with a different fluorescent primer to allow their discrimination when analyzed by gel or capillary electrophoresis using an automated DNA Analysis System such as the PE Biosystems Models 310/373/377 or 3700.

SNPs also can be genotyped rapidly and efficiently using techniques that make use of thermal denaturation differences due to differences in DNA base composition. In one embodiment of this test, allele specific primers are designed as above to detect biallelic SNP with the exception that to one primer is added a 5′ GC tail of 26 bases (Germer and Higuichi, 1999). After PCR amplification with a single, common reverse primer, a fluorescent dye that binds preferentially to dsDNA (e.g., SYBR Green 1) is added to the tube and then the thermal denaturation profile of the dsDNA product of PCR amplification is determined. Samples homozygous for the SNP amplified by the GC tailed primer will denature at the high end of the temperature scale, while samples homozygous for the SN amplified by the non-GC tagged primer will denature at the low end of the temperature scale. Heterozygous samples will show two peaks in the thermal denaturation profile.

In a variant of the foregoing technique, dynamic allele-specific hybridization (DASH) is detected by thermal denaturation curves (Howell et al., 1999). In on embodiment of this test, a pair of PCR primers is used to amplify the genomic region in the DNA sample containing the SNP. One of these primers is biotinylated to allow subsequent binding of the biotinylated product strand to strepavidin-coated microtiter plates while the non-biotinylated strand is washed away with alkali. An oligoucleotide probe which is an exact match for one allele is hybridized to the immobilized PCR product at low temperature. This forms a dsDNA region that interacts with a dsDNA intercalating dye (e.g., SYBR Green 1). The thermal denaturation profile then allows the test to distinguish the single base mismatch between the biallelic SNP due to the difference in melting temperature. Other methods for SNP genotyping and their application to the detection of SNP in the Con-202 gene can be envisaged by one skilled in the art.

### EXAMPLE 2

### Genotyping a Schizophrenic Population

Objectives

To genotype DNA from the NIMH schizophrenia samples for the SNPs found in and around the gene for our orphan G protein-coupled receptor Con-202 to see if Con-202 is associated with an increase risk for schizophrenia.

Materials and Methods

DNA Samples

DNA samples were obtained from National Institute of Mental Health Schizophrenia Genetics Initiative. Twenty-five nanograms from each DNA sample was placed in 96 well plates (Schz01, Schz02, Schz03, Schz04), dried down, then stored at −20° C. The plates labeled Schz01, Schz02 and Schz03 contained 72 samples with the first three columns left blank to be used for the controls. The plate labeled Schz04 contained 9 samples in wells A4 to A12.

Allelic Discrimination using TaqMan® MGB Probes

Primers and Taqman® MGB probes (Applied Biosystems) for each SNP were designed using the software Primer Express version 1.5 (Applied Biosystems). Table 3 list all the primers and probes for each SNP. The primers were resuspended in H₂O at a final concentration of 100 μM. The PCR reaction was done in 10 μl consisting of the following: 5 μl of 2×TaqMan® Universal PCR Master Mix, 200 nM of each probe, 900 nM of the forward and reverse primers, and H₂O to 10 μl. The 10 μl was added to the dried down DNA samples. The following controls were done on each plate, 8 no template controls, 8 allele 1 controls and 8 allele 2 controls. The allele 1 and 2 controls contained 25 ng of DNA from the Con01 described in Example 1. The plates were placed on a Titer Plate Shaker and shaken vigorously for 5 minutes then briefly spun at 1000 rpm. Thermal cycling was performed in a 9600 cycler (Applied Biosystems) with the following thermal cycling conditions: 50° C. for 2 min→95° C. for 10 min→35 cycles of 92° C. for 15 sec, 60° C. for 1 min. The fluorescent signal was detected using an ABI PRISM 7700 Sequence Detector (Applied Biosystems) following the manufactures instructions for an endpoint plate read. The data was analyzed with the SDS software version 1.7 (Applied Biosystems).

Results

To do allelic discrimination on the schizophrenia samples we are using the TaqMan® assay method. This method involves designing two probes, one that contains the more common allele (allele 1) and another probe that contains the least common allele (allele 2). The two probes contain different fluorescent reporter dyes (FAM and VIC), which are used to differentiate the two alleles, and a nonflurescent quencher dye. Forward and reverse primers are designed flanking the probe to give a PCR product between 75 and 150 bp. The two probes and the primers are added to the DNA in a PCR assay. If the DNA sample is homozygous for allele 1 the probe for allele 1 will hybridize to the PCR product, the reporter dye will be cleaved by the 5′ nuclease activity of Taq DNA polymerase, and there will be an increase fluorescense of that reporter dye. If the DNA sample is homozygous for allele 2 there will be an increase fluorescence of the reporter dye attached to the allele 2 probe. If the sample is heterozygous for allele 1 and 2 there will be an increase fluorescence in both reporter dyes. Following the PCR reaction the fluorescence is read on an ABI PRISM 7700 Sequence Dectector. The primers and TaqMan® MGB probes used for allelic discrimination are shown in Table 3. The table indicates which probe contains the allele 1 SNP or the allele 2 SNP. For SNPs #1,2 and 7 the probes are designed for the sense strand and for SNPs #3 and 4 the probes are for the antisense (complementary) strand. In Table 3, the SNP nucleotide is in bold type and underlined. All (allele 1) indicates the probe contains the SNP for the more common allele and A12 (allele 2) indicates the probe contains the SNP for the rare allele. F is the forward primer and R is the reverse.

TABLE 3

Primers and TaqMan ® MGB probes for Con-202

| | | |
|---|---|---|
| SNP | Tagman MGB Probes (concentration) | Primers |

| | | | |
|---|---|---|---|
| S1 | Al2 6FAM-ATGCACTTCCATTTGTA-MGVNFQ | (100 uM) | F:AAATACAGGTTCATGTTATATTCATTTGAATC |
| | SEQ ID NO:6 | SEQ ID NO:16 | |
| | Al1:VIC-ATGCACTTGCATTTG-MGBNFQ | (94 uM) | R:TTAATGTAAATATTGGACAGGGAAACAC |
| | SEQ ID NO:7 | SEQ ID NO:17 | |
| S2 | Al1: 6FAM-AGGCTGTATCCTTATC-MGBNFQ | (100 uM) | F:ATGGATCTATCTTTTCCTTGITCTTATATCTA |
| | SEQ ID NO:8 | SEQ ID NO:18 | |
| | Al2:VIC-AGGCTGTATACTTATC-MGBNFQ | (100 uM) | R:TGTCAGCTGCATGGCTATAGTTC |
| | SEQ ID NO:9 | SEQ ID NO:19 | |
| S3 | Al1:6FAM-CCAGGTAGAGCCATT-MGBNFQ | (77 uM) | F:TTGTTTCCCATTTGTGTTCAACTC |
| | SEQ ID NO:10 | SEQ ID NO:20 | |
| | Al2:VIC-TCCAGGTAGAACCATT-MGBNFQ | (85 uM) | R:AGAAAGGCAATCACTTTGCAAGTC |
| | SEQ ID NO:11 | SEQ ID NO:21 | |
| S4 | Al1:6FAM-CGATAGCTAAGTATCTG-MGBNFQ | (95 uM) | F:TCCTGTTTCCACACTGCTTTCA |
| | SEQ ID NO:12 | SEQ ID NO:22 | |
| | Al2:VIC-CGATAGCTAAATATCTG-MGBNFQ | (94 uM) | R:AGCCAGACACGTCCAAAAGG |
| | SEQ ID NO:13 | SEQ ID NO:23 | |
| S7 | Al1:GFAM-CAAAGCAGTATATTGGT-MGBNFQ | (71 uM) | F:GTGCCAATGAATTATTGGAATATCAC |
| | SEQ ID NO:14 | SEQ ID NO:24 | |
| | Al2:VIC-CAAAGCAGTGTATTGGT-MGBNFQ | (89 uM) | R:GAGGAAAGTGGTATTTAGAAATAGCAATG |
| | SEQ ID NO:15 | SEQ ID NO:25 | |

The experiment was designed to have eight no template controls, eight known allele 1 controls and eight known allele 2 controls on each plate which the SDS software uses to autocall the results of the unknown samples. The control DNA came from the samples used during SNP discovery (Example 1) except for allele 2 of SNPs #3 and 4. For these two SNPs no allele 2 was found during discovery but the genomic clone we isolated had the allele 2 version of both of these SNPs. Twenty-five micrograms of DNA from the 225 NIMH schizophrenia samples was aliquoted to the remaining wells of a 96 well microtiter plate then dried down.

The demographics of the individuals in the NIMH samples we obtained is well defined We collected genotype data from 225 individuals (however 248 were available for statistical analysis detailed in Exmaple 3) from 62 nuclear families selected for our study. There were 53 individual with unknown maternal ethnicity, roughly 68 individuals' mother's were African American, 51 were West European, 39 were Anglo-Saxon, 14 were Mediterranean and 23 with other ethnic background (numbers not entirely additive) A total of 111 individuals were diagnosed with schizophrenia, 50 individuals never had mental illness, 15 individuals had unknown disease statuses and 72 individuals had different types of mental illness.

The TaqMan® assay was performed for five SNPs on the 225 NIMH schizophrenia samples and genotype data collected for each individual. Each individual was either homozygous or heterozygous for each allele.

Table 4 gives a summary of the frequencies of the rare allele from both the NIMH samples and from the CON01 samples that were used in the SNP discovery phase. Any differences between the two could be due to the difference in sample size (72 from CON01 vs 225 from NIMH). (It should be noted that the percentages below are based on a total sample size of 225 for the NIMH samples and not all these individuals were diagnosed as schizophrenic. Therefore., the results obtained were analyzed further for significance as described in Example 3 below (and disease state was taken into account for this analysis)

TABLE 4
Summary of genotyping results. The percentage is of the rare allele as
determined during the discovery phase.

| Con-202 SNP# | 1 | 2 | 3 | 4 | 7 |
|---|---|---|---|---|---|
| NIMH SZ | 5.40% | 22.60% | 11.10% | 10.40% | 31.80% |
| discovery (CON01) | 13.90% | 21.10% | 10.90% | 10.90% | 37% |

Polymorphisms of the Invention in Methods of Genetic Diagnostics

The polymorphisms of the present invention can also be used to develop diagnostics tests capable of identifying individuals who are at increased risk of developing schizophrenia or suffers from schizophrenia. The diagnostic techniques of the present invention may employ a variety of methodologies to determine whether a test subject has a polymorphic marker pattern associated with an increased risk of developing schizophrenia or whether the individual suffers from schizophrenia coincident with carrying a particular mutation, including methods which enable the analysis of individual chromosomes for haplotyping, such as family studies, single sperm DNA analysis or somatic hybrids.

Determining the Haplotype of an Individual

It will be apparent from Example 3 below that it is particularly advantageous to determine the identity of nucleotides occupying specific polymorphic sites on the same chromosomal segment in an individual (the haplotype).

The present invention therefore further provides a method of diagnosing a schizophrenia or determining a predisposition to schizophrenia by determining the presence or absence of a Con-202 haplotype in a patient by obtaining material comprising nucleic acid including the polymorphic sites at position, 184, 1022, 1292, 1400 and 2724 of SEQ ID NO:1 from the patient; enzymatically amplifying the nucleic acid using pairs of oligonucleotide primers complementary to nucleotide sequences flanking any of the polymorphic sites at position, 184, 1022, 1292, 1400 and 2724 of SEQ ID NO:1 to produce amplified products containing any of the polymorphic site or other Con-202 polymorphic sites and determining the Con-202 haplotype.

In order to determine a haplotype one skilled in the art understands that an amplified product can be sequenced directly or subcloned into a vector prior to sequence analysis. Commercially available sequencing kits including the Sequenase TM kit from Amersham Life Science (Arlington Heights, Ill.) can be used to sequence an amplified product in the methods of the invention. Automated sequence analysis also can be useful, and automated sequencing instruments such as the Prism 377 DNA Sequencer or the 373 DNA Sequencer are commercially available, for example, from Applied Biosystems (Foster City, Calif.; see, also, Frazier et al., Electrophoresis 17:1550-1552 (1996), which is incorporated herein by reference). Both copies in a diploid genome give rise to sequence the haplotypic composition of an individual can thus be inferred from direct sequence analysis.

Another possibility is that single chromosomes can be studied independently, for example, by asymmetric PCR amplification (see Newton et al., Nucleic Acids Res., 17:2503-2516, 1989; Wu et al., Proc. Natl Acad Sci. USA, 86:2757, 1989) or by isolation of single chromosome by limit dilution followed by PCR amplification (see Ruano et al., Proc. Natl. Acad. Sci. USA, 87:6296-6300, 1990). Further, a sample may be haplotyped for sufficiently close polymorphic markers by double PCR amplification of specific alleles (Sarkar, G. and Sommer S. S., Biotechniques, 1991).

The present invention provides diagnostic methods to determine whether an individual is at risk of developing schizophrenia or suffers from schizophrenia coincident with a mutation or a polymorphism in of the present invention. The present invention also provides methods to determine whether an individual is likely to respond positively to an agent acting on schizophrenia disorder or whether an individual is at risk of developing an adverse side effect to an agent acting on schizophrenia

These methods involve obtaining a nucleic acid sample from the individual and, determining, whether the nucleic acid sample contains at least one allele or at least one polymorphic haplotype, indicative of a risk of developing the trait or indicative that the individual expresses the trait as a result of possessing trait-causing allele.

Preferably, in such diagnostic methods, a nucleic acid sample is obtained from the individual and this sample is genotyped using methods described above. The diagnostics may be based on a single polymorphism or on a group of polymorphisms. In each of these methods, a nucleic acid sample is obtained from the test subject and the polymorphic pattern of one or more of the polymorphic markers listed in Table 1 and 2 is determined.

In one embodiment, PCR amplification is conducted on the nucleic acid sample to amplify regions in which polymorphisms associated with a detectable phenotype have been identified. The amplification products are sequenced to determine whether the individual possesses one or more polymorphisms associated with a detectable phenotype. The primers used to generate amplification products may comprise the primers listed in Table 3. Alternatively, the nucleic acid sample is subjected to microsequencing reactions as described above to determine whether the individual possesses one or more polymorphisms associated with a detectable phenotype resulting from a mutation or a polymorphism in a candidate gene. The primers used in the microsequencing reactions may include the primers listed in Table 3. In another embodiment, the nucleic acid sample is contacted with one or more allele specific oligonucleotide probes which, specifically hybridize to one or more candidate gene alleles associated with a detectable phenotype. The probes used in the hybridization assay may include the probes listed in Table 3

In a preferred embodiment the identity of the nucleotide present at, at least one, biallelic marker selected from the group consisting the polymorphic sites at position, 184, 1022, 1292, 1400 and 2724 of SEQ ID NO:1, is determined and the detectable trait is schizophrenia.

These diagnostic methods are extremely valuable as they can, in certain circumstances, be used to initiate preventive treatments or to allow an individual carrying a significant haplotype to foresee warning signs such as minor symptoms. In diseases in which attacks may be extremely violent and sometimes fatal if not treated on time, such as asthma, the knowledge of a potential predisposition, even if this predisposition is not absolute, might contribute in a very significant manner to treatment efficacy. Similarly, a diagnosed predisposition to a potential side effect could immediately direct the physician toward a treatment for which such side effects have not been observed during clinical trials.

Diagnostics, which analyze and predict response to a drug or side effects to a drug, may be used to determine whether an individual should be treated with a particular drug. For example, if the diagnostic indicates a likelihood that an individual will respond positively to treatment with a particular drug, the drug may be administered to the individual. Conversely, if the diagnostic indicates that an individual is likely to respond negatively to treatment with a particular drug, an alternative course of treatment may be prescribed. A negative response may be defined as either the absence of an efficacious response or the presence of toxic side effects.

Clinical drug trials represent another application for the markers of the present invention. One or more markers indicative of response to an agent acting on schizophrenia or to side effects to an agent acting on schizophrenia may be identified using the methods described above. Thereafter, potential participants in clinical trials of such an agent may be screened to identify those individuals most likely to respond favorably to the drug and exclude those likely to experience side effects. In that way, the effectiveness of drug treatment may be measured in individuals who respond positively to the drug, without lowering the measurement as a result of the inclusion of individuals who are unlikely to respond positively in the study and without risking undesirable safety problems.

Diagnostic Kits Comprising Polynucleotides of the Present Invention are Further Described Below

Diagnostic Kits

The invention further provides kits comprising at least one allele-specific oligonucleotide as described above. Often, the kits contain one or more pairs of allele-specific oligonucleotides hybridizing to different forms of a polymorphism. In some kits, the allele-specific oligonucleotides are provided immobilized to a substrate. For example, the same substrate can comprise allele-specific oligonucleotide probes for detecting both of the polymorphisms described. Optional additional components of the kit include, for example, restriction enzymes, reverse-transcriptase or polymerase, the substrate nucleoside triphosphates, means used to label (for example, an avidinenzyme conjugate and enzyme substrate and chromogen if the label is biotin), and the appropriate buffers for reverse transcription, PCR, or hybridization reactions. Usually, the kit also contains instructions for carrying out the methods The present invention is used to determine whether or not an individual has an Con-202 polymorphism which has been associated with schizophrenia. Such Con-202 polymorphisms are shown to be genetic risk factors in population studies which compare the frequency of the said polymorphism in the general population and the frequency of the polymorphism in persons with schizophrenia. If for example, said polymophism occurs at a frequency of 3% in the general population, but at a frequency of 30% in persons with schizophrenia, then a test for said polymorphism will reveal individuals having a higher risk for developing schizophrenia. This information may be used either prognositically to identify individuals with increased risk for developing schizophrenia at a future point in time, or diagnostically to identify individuals presenting with schizophrenia on clinical exam who may therefore be diagnosed as being more likely to have schizophrenia, or other related diseases such as schizoaffective disorder-bipolar, schizoaffective disorder-depression, schizotypal personality disorder, non-affective psychotic disorder (schizophreniform disorder, delusional disorder, psychotic disorder NOS), or mood-incongruent psychotic depressive disorder or paranoid or schizoid personality disorder.

Analysis of said Con-202 polymorphism for the purpose of prognosis or diagnosis may be performed by one of any techniques capable of accurately detecting SNP including but not limited to allele-specific hybridization on filters, allele-specific PCR, PCR plus restriction enzyme digest (RFLP-PCR), denaturing capillary electrophoresis, primer extension and time-of-flight mass spectrometry, and the 5′ nuclease (Taq-Man) assay.

Preferred techniques for SNP genotyping should allow large scale, automated analysis which do not require extensive optimization for each SNP analyzed. Examples of the later are DASH (Dynamic Allele-Specific hybridization) which is amenable to formatting in microtiter plates (Hybaid) and “single-stringency” DNA-chip hybridization (Affymetrix).

Methods of Genetic Analysis Using the Polymorphic Markers of the Present Invention

Once the identity of a polymorphism has been established it becomes desirable to attempt to associate a particular form of the polymorphism with the presence or absence of a phenotype. It is apparent that while we have established an association of certain polymorphisms of the invention with a schizophrenia phenotype, the invention also contemplates the use of the polymorphic sites of the invention as markers for the analysis of other disease states, of suceptibility to drug treatment for schizophrenia or other diseases, or may be included in any complete or partial genetic map of the human genome

The polymorphic markers of the present invention find use in any method known in the art to demonstrate a statistically significant correlation between a genotype and a phenotype. Different methods are available for the genetic analysis of complex traits (see Lander and Schork, Science, 265, 2037-2048, 1994). To determine if a polymorphism is associated with a phenotypic trait three main methods are used: the linkage approach (either parametric or non-parametric) in which evidence is sought for cosegregation between a locus and a putative trait locus using family studies, and the association approach in which evidence is sought for a statistically significant association between an allele and a trait or a trait causing allele and the TDT approach which tests for both linkage and association

The polymorphic markers may be used in parametric and non-parametric linkage analysis methods. Preferably, the polymorphic markers of the present invention are used to identify genes associated with schizophrenia or other disorders using association studies such as the case control method, an approach which does not require the use of affected families and which permits the identification of genes associated with complex and sporadic traits.

The genetic analysis using the polymorphic markers of the present invention may be conducted on any scale. The whole set of polymorphic markers of the present invention or any subset of polymorphic markers of the present invention may be used. Further, any set of genetic markers including a polymorphic marker of the present invention may be used. A set of biallelic polymorphisms that, could be used as genetic markers in combination with the polymorphic markers of the present invention, has been described in WO 98/20165. As mentioned above, it should be noted that the polymorphic markers of the present invention may be included in any complete or partial genetic map of the human genome.

These different uses are specifically contemplated in the present invention and claims.

A. Linkage Analysis

Linkage analysis is based upon establishing a correlation between the transmission of genetic markers and that of a specific trait throughout generations within a family. Thus, the aim of linkage analysis is to detect marker loci that show cosegregation with a trait of interest in pedigrees.

Parametric methods

When data are available from successive generations there is the opportunity to study the degree of linkage between pairs of loci. Estimates of the recombination fraction enable loci to be ordered and placed onto a genetic map. With loci that are genetic markers, a genetic map can be established, and then the strength of linkage between markers and traits can be calculated and used to indicate the relative positions of markers and genes affecting those traits. The classical method for linkage analysis is the logarithm of odds (lod) score method (see Morton N. E., Am. J Hum. Genet., 7:277-318, 1955; Ott J., Analysis of Human Genetic Linkage, John Hopkins University Press, Baltimore, 1991). Calculation of lod scores requires specification of the mode of inheritance for the disease (parametric method). Generally, the length of the candidate region identified using linkage analysis is between 2 and 20 Mb. Once a candidate region is identified as described above, analysis of recombinant individuals using additional markers allows further delineation of the candidate region. Linkage analysis studies have generally relied on the use of a maximum of 5,000 microsatellite markers, thus limiting the maximum theoretical attainable resolution of linkage analysis to about 600 kb on average.

Linkage analysis has been successfully applied to map simple genetic traits that show clear Mendelian inheritance patterns and which have a high penetrance (i.e., the ratio between the number of trait positive carriers of allele and the total number of a carriers in the population). However, parametric linkage analysis suffers from a variety of drawbacks. First, it is limited by its reliance on the choice of a genetic model suitable for each studied trait. Furthermore, as already mentioned, the resolution attainable using linkage analysis is limited, and complementary studies are required to refine the analysis of the typical 2 Mb to 20 Mb regions initially identified through linkage analysis. In addition, parametric linkage analysis approaches have proven difficult when applied to complex genetic traits, such as those due to the combined action of multiple genes and/or environmental factors. It is very difficult to model these factors adequately in a lod score analysis. In such cases, too large an effort and cost are needed to recruit the adequate number of affected families required for applying linkage analysis to these situations, as recently discussed by Risch, N. and Merikangas, K. (Science, 273:1516-1517, 1996).

Non-Parametric Methods

The advantage of the so-called non-parametric methods for linkage analysis is that they do not require specification of the mode of inheritance for the disease, they tend to be more useful for the analysis of complex traits. In non-parametric methods, one tries to prove that the inheritance pattern of a chromosomal region is not consistent with random Mendelian segregation by showing that affected relatives inherit identical copies of the region more often than expected by chance. Affected relatives should show excess “allele sharing” even in the presence of incomplete penetrance and polygenic inheritance. In non-parametric linkage analysis the degree of agreement at a marker locus in two individuals can be measured either by the number of alleles identical by state (IBS) or by the number of alleles identical by descent (IBD). Affected sib pair analysis is a well-known special case and is the simplest form of these methods.

The polymorphic markers of the present invention may be used in both parametric and non-parametric linkage analysis. Preferably polymorphic markers may be used in non-parametric methods which allow the mapping of genes involved in complex traits. The polymorphic markers of the present invention may be used in both IBD-and IBS— methods to map genes affecting a complex trait. In such studies, taking advantage of the high density of polymorphic markers, several adjacent polymorphic marker loci may be pooled to achieve the efficiency attained by multi-allelic markers (Zhao et al., Am. J. Hum. Genet., 63:225-240,1998).

However, both parametric and non-parametric linkage analysis methods analysis require access affected relatives, they tend to be of limited value in the genetic analysis of drug responses or in the analysis of side effects to treatments. This type of analysis is impractical in such cases due to the lack of availability of familial cases. In fact, the likelihood of having more than one individual in a family being exposed to the same drug at the same time is extremely low.

B. Population Association Studies

The present invention comprises methods for identifying polymorphic markers that are associated with a detectable trait using the polymorphic markers of the present invention. In one embodiment the present invention comprises methods to detect an association between a polymorphic marker allele or a polymorphic marker haplotype and a trait. Further, the invention comprises methods to identify a trait causing allele in linkage disequilibrium with any polymorphic marker allele of the present invention.

As mentioned above, association studies may be conducted within the general population and are not limited to studies performed on related individuals in affected families. Association studies are extremely valuable as they permit the analysis of sporadic or multifactor traits. Moreover, association studies represent a powerful method for fine-scale mapping enabling much finer mapping of trait causing alleles than linkage studies. Studies based on pedigrees often only narrow the location of the trait causing allele. Association studies using the polymorphic markers of the present invention can therefore be used to refine the location of a trait causing allele in a candidate region identified by Linkage Analysis methods. Polymorphic markers of the present invention can be used to demonstrate that a particular gene is associated with a trait. Such uses are specifically contemplated in the present invention and claims.

The general strategy to perform association studies using polymorphic markers is to scan two groups of individuals (case-control populations) in order to measure and statistically compare the allele frequencies of the polymorphic markers of the present invention in both groups.

If a statistically significant association with a trait is identified for at least one or more of the analyzed polymorphic markers, one can assume that: either the associated allele is directly responsible for causing the trait (the associated allele is the trait causing allele), or more likely the associated allele is in linkage disequilibrium with the trait causing allele. The specific characteristics of the associated allele with respect to the candidate gene function usually gives further insight into the relationship between the associated allele and the trait (causal or in linkage disequilibrium). If the evidence indicates that the associated allele within the candidate gene is most probably not the trait causing allele but is in linkage disequilibrium with the real trait causing allele, then the trait causing allele can be found by sequencing the vicinity of the associated marker.

Association studies are usually run in two successive steps. In a first phase, the frequencies of several polymorphic markers are determined in the trait positive and trait negative populations. In a second phase of the analysis, the identity of the candidate gene and the position of the genetic loci responsible for the given trait is further refined using a higher density of markers from the relevant region.

Haplotype Analysis

As described above, when a chromosome carrying a disease allele first appears in a population as a result of either mutation or migration, the mutant allele necessarily resides on a chromosome having a set of linked markers: the ancestral haplotype. This haplotype can be tracked through populations and its statistical association with a given trait can be analyzed. Complementing single point (allelic) association studies with multi-point association studies also called haplotype studies increases the statistical power of association studies. Thus, a haplotype association study allows one to define the frequency and the type of the ancestral carrier haplotype. A haplotype analysis is important in that it increases the statistical power of an analysis involving individual markers.

In a first stage of a haplotype frequency analysis, the frequency of the possible haplotypes based on various combinations of the identified polymorphic markers of the invention is determined. The haplotype frequency is then compared for distinct populations of trait positive and control individuals. The number of trait positive individuals, which should be, subjected to this analysis to obtain statistically significant results usually ranges between 30 and 300, with a preferred number of individuals ranging between 50 and 150. The same considerations apply to the number of unaffected individuals (or random control) used in the study. The results of this first analysis provide haplotype frequencies in case-control populations, for each evaluated haplotype frequency a p-value and an odd ratio are calculated. If a statistically significant association is found the relative risk for an individual carrying the given haplotype of being affected with the trait under study can be approximated.

Interaction Analysis

The polymorphic markers of the present invention may also be used to identify patterns of polymorphic markers associated with detectable traits resulting from polygenic interactions. The analysis of genetic interaction between alleles at unlinked loci requires individual genotyping using the techniques described herein. The analysis of allelic interaction among a selected set of polymorphic markers with appropriate level of statistical significance can be considered as a haplotype analysis. Interaction analysis consists of stratifying the case-control populations with respect to a given haplotype for the first loci and performing a haplotype analysis with the second loci with each subpopulation.

Statistical methods used in association studies are further described below.

1.) Determining the Frequency of a Polymorphic Marker Allele or of a Polymorphic Marker Haplotype in a Population

Determining the Frequency of an Allele in a Population

Allelic frequencies of the polymorphic markers in a population can be determined using one of the methods described above or any genotyping procedure suitable for this intended purpose. Genotyping pooled samples or individual samples can determine the frequency of a polymorphic marker allele in a population. One way to reduce the number of genotypings required is to use pooled samples. A major obstacle in using pooled samples is in terms of accuracy and reproducibility for determining accurate DNA concentrations in setting up the pools. Genotyping individual samples provides higher sensitivity, reproducibility and accuracy and; is the preferred method used in the present invention. Preferably, each individual is genotyped separately and simple gene counting is applied to determine the frequency of an allele of a polymorphic marker or of a genotype in a given population.

Determining the Frequency of a Haplotype in a Population

The gametic phase of haplotypes is unknown when diploid individuals are heterozygous at more than one locus. Using genealogical information in families gametic phase can sometimes be inferred (Perlin et al., Am. J. Hum. Genet., 55:777-787, 1994). When no genealogical information is available different strategies may be used. One possibility is that the multiple-site heterozygous diploids can be eliminated from the analysis, keeping only the homozygotes and the single-site heterozygote individuals, but this approach might lead to a possible bias in the sample composition and the underestimation of low-frequency haplotypes. As noted earlier, another possibility is that single chromosomes can be studied independently, for example, by asymmetric PCR amplification (see Newton et al., Nucleic Acids Res., 17:2503-2516, 1989; Wu et al., Proc. Natl. Acad. Sci. USA, 86:2757, 1989) or by isolation of single chromosome by limit dilution followed by PCR amplification (see Ruano et al., Proc. Natl. Acad. Sci. USA, 87:6296-6300, 1990). Further, a sample may be haplotyped for sufficiently close polymorphic markers by double PCR amplification of specific alleles (Sarkar, G. and Sommer S. S., Biotechniques, 1991). These approaches are not entirely satisfying either because of their technical complexity, the additional cost they entail, their lack of generalization at a large scale, or the possible biases they introduce. To overcome these difficulties, an algorithm to infer the phase of PCR-amplified DNA genotypes introduced by Clark A. G. (Mol Biol Evol, 7:111-122, 1990) may be used. Briefly, the principle is to start filling a preliminary list of haplotypes present in the sample by examining unambiguous individuals, that is, the complete homozygotes and the single-site heterozygotes. Then other individuals in the same sample are screened for the possible occurrence of previously recognized haplotypes. For each positive identification, the complementary haplotype is added to the list of recognized haplotypes, until the phase information for all individuals is either resolved or identified as unresolved. This method assigns a single haplotype to each multiheterozygous individual, whereas several haplotypes are possible when there are more than one heterozygous site. Alternatively, one can use methods estimating haplotype frequencies in a population without assigning haplotypes to each individual. Preferably, a method based on an expectation-maximization (EM) algorithm (Dempster et al., J R. Stat. Soc., 39B: 1-38, 1977) leading to maximum-likelihood estimates of haplotype frequencies under the assumption of Hardy-Weinberg proportions (random mating) is used (see Excoffier L. and Slatkin M., Mol Biol Evol, 12(5): 921927, 1995). The EM algorithm is a generalized iterative maximum-likelihood approach to estimation that is useful when data are ambiguous and/or incomplete. Haplotype estimations are further described below under the heading “Statistical methods”. Any other method known in the art to determine or to estimate the frequency of a haplotype in a population may also be used.

2. Linkage Disequilibrium Analysis

Linkage disequilibrium is the non-random association of alleles at two or more loci and represents a powerful tool for mapping genes involved in disease traits (see Ajioka R. S. et al., Am. J Hum. Genet., 60:1439-1447,1997). Polymorphic markers, because they are densely spaced in the human genome and can be genotyped in more numerous numbers than other types of genetic markers (such as RFLP or VNTR markers), are particularly useful in genetic analysis based on linkage disequilibrium. The polymorphic markers of the present invention may be used in any linkage disequilibrium analysis method known in the art.

Briefly, when a disease mutation is first introduced into a population (by a new mutation or the immigration of a mutation carrier), it necessarily resides on a single chromosome and thus on a single “background” or “ancestral” haplotype of linked markers. Consequently, there is complete disequilibrium between these markers and the disease mutation: one finds the disease mutation only in the presence of a specific set of marker alleles. Through subsequent generations recombinations occur between the disease mutation and these marker polymorphisms, and the disequilibrium gradually dissipates. The pace of this dissipation is a function of the recombination frequency, so the markers closest to the disease gene will manifest higher levels of disequilibrium than those further away. When not broken up by recombination, “ancestral” haplotypes and linkage disequilibrium between marker alleles at different loci can be tracked not only through pedigrees but also through populations. Linkage disequilibrium is usually seen as an association between one specific allele at one locus and another specific allele at a second locus.

The pattern or curve of disequilibrium. between disease and marker loci is expected to exhibit a maximum that occurs at the disease locus. Consequently, the amount of linkage disequilibrium. between a disease allele and closely linked genetic markers may yield valuable information regarding the location of the disease gene. For fine-scale mapping of a disease locus, it is useful to have some knowledge of the patterns of linkage disequilibrium that exist between markers in the studied region. As mentioned above the mapping resolution achieved through the analysis of linkage disequilibrium is much higher than that of linkage studies. The high density of polymorphic markers combined with linkage disequilibrium analysis provides powerful tools for fine-scale mapping.

Once a first polymorphic marker has been identified in a genomic region of interest, the practitioner of ordinary skill in the art, using the teachings of the present invention, can easily identify additional polymorphic markers in linkage disequilibrium with this first marker. As mentioned before any marker in linkage disequilibrium with a first marker associated with a trait will be associated with the trait. Therefore, once an association has been demonstrated between a given polymorphic marker and a trait, the discovery of additional polymorphic markers associated with this trait is of great interest in order to increase the density of polymorphic markers in this particular region. The causal gene or mutation will be found in the vicinity of the marker or set of markers showing the highest correlation with the trait.

Identification of additional markers in linkage disequilibrium with a given marker involves: (a) amplifying a genomic fragment comprising a first polymorphic marker from a plurality of individuals; (b) identifying of second polymorphic markers in the genomic region harboring said first polymorphic marker; (c) conducting a linkage disequilibrium analysis between said first polymorphic marker and second polymorphic markers; and (d) selecting said second polymorphic markers as being in linkage disequilibrium with said first marker. Subcombinations comprising steps (b) and (c) are also contemplated.

Methods to identify polymorphic markers and to conduct linkage disequilibrium analysis are described herein and can be carried out by the skilled person without undue experimentation. The present invention then also concerns polymorphic markers which are in linkage disequilibrium with the specific polymorphic markers shown in Table 1 and which are expected to present similar characteristics in terms of their respective association with a given trait.

Different methods to calculate linkage disequilibrium are described below under the heading “Statistical Methods”.

3. Population-Based Case-Control Studies of Trait-Marker Associations

As mentioned above, the occurrence of pairs of specific alleles at different loci on the same chromosome is not random and the deviation from random is called linkage disequilibrium. Association studies focus on population frequencies and rely on the phenomenon of linkage disequilibrium. If a specific allele in a given gene is directly involved in causing a particular trait, its frequency will be statistically increased in an affected (trait positive) population, when compared to the frequency in a trait negative population or in a random control population. As a consequence of the existence of linkage disequilibrium, the frequency of all other alleles present in the haplotype carrying the trait-causing allele will also be increased in trait positive individuals compared to trait negative individuals or random controls. Therefore, association between the trait and any allele (specifically a polymorphic marker allele) in linkage disequilibrium with the trait-causing allele will suffice to suggest the presence of a trait-related gene in that particular region. Case-control populations can be genotyped for polymorphic markers to identify associations that narrowly locate a trait causing allele. As any marker in linkage disequilibrium with one given marker associated with a trait will be associated with the trait. Linkage disequilibrium allows the relative frequencies in case-control populations of a limited number of genetic polymorphisms (specifically polymorphic markers) to be analyzed as an alternative to screening all possible functional polymorphisms in order to find trait-causing alleles. Association studies compare the frequency of marker alleles in unrelated case-control populations, and represent powerful tools for the dissection of complex traits.

Case-Control Populations (Inclusion Criteria)

Population-based association studies do not concern familial inheritance but compare the prevalence of a particular genetic marker, or a set of markers, in case-control populations. They are case-control studies based on comparison of unrelated case (affected or trait positive) individuals and unrelated control (unaffected or trait negative or random) individuals. Preferably the control group is composed of unaffected or trait negative individuals. Further, the control group is ethnically matched to the case population. Moreover, the control group is preferably matched to the case-population for the main known confusion factor for the trait under study (for example age-matched for an age-dependent trait). Ideally, individuals in the two samples are paired in such a way that they are expected to differ only in their disease status. In the following “trait positive population”, “case population” and “affected population” are used interchangeably.

An important step in the dissection of complex traits using association studies is the choice of case-control populations (see Lander and Schork, Science, 265, 2037-2048, 1994). A major step in the choice of case-control populations is the clinical definition of a given trait or phenotype. Any genetic trait may be analyzed by the association method proposed here by carefully selecting the individuals to be included in the trait positive and trait negative phenotypic groups. Four criteria are often useful: clinical phenotype, age at onset, family history and severity. The selection procedure for continuous or quantitative traits (such as blood pressure for example) involves selecting individuals at opposite ends of the phenotype distribution of the trait under study, so as to include in these trait positive and trait negative populations individuals with non-overlapping phenotypes. Preferably, case-control populations consist of phenotypically homogeneous populations. Trait positive and trait negative populations consist of phenotypically uniform populations of individuals representing each between 1 and 98%, preferably between 1 and 80%, more preferably between 1 and 50%, and more preferably between 1 and 30%, most preferably between 1 and 20% of the total population under study, and selected among individuals exhibiting non-overlapping phenotypes. The clearer the difference between the two trait phenotypes, the greater the probability of detecting an association with polymorphic markers. The selection of those drastically different but relatively uniform phenotypes enables efficient comparisons in association studies and the possible detection of marked differences at the genetic level, provided that the sample sizes of the populations under study are significant enough.

In preferred embodiments, a first group of between 50 and 300 trait positive individuals, preferably about 100 individuals, are recruited according to their phenotypes. A similar number of trait negative individuals are included in such studies.

In the present invention, typical examples of inclusion criteria include a CNS disorder or the evaluation of the response to a drug acting on a CNS disorder or side effects to treatment with drugs acting on a CNS disorder.

Suitable examples of association studies using polymorphic markers including the polymorphic markers of the present invention, are studies involving the following populations:

1. a case population treated with agents acting on schizophrenia suffering from side-effects resulting from the treatment and a control population treated with the same agents showing no side-effects, or

2. a case population treated with agents acting on schizophrenia showing a beneficial response and a control population treated with same agents showing no beneficial response.

3. a case population suffering from a another CNS disorder and a healthy unaffected control population.

C. Testing for Linkage in the Presence of Association

The polymorphic markers of the present invention may further be used in TDT (transmission/disequilibrium test). TDT tests for both linkage and association and is not affected by population stratification. TDT requires data for affected individuals and their parents or data from unaffected sibs instead of from parents (see Spielman. S. et al., Am. J Hum. Genet., 52:506-516,1993; Schaid D. J. et al., Genet. Epidemiol.,13:423-450, 1996, Spielman. S. and Ewens W. J., Am. J Hum. Genet., 62:450-458,1998). This method employs a family-based experimental design to avoid potential pitfalls due to mismatching of case and control groups or admixture of subpopulations consisting of different racial or ethnic groups. In addition, theoretical analyses indicate that this approach may be much more powerful than traditional linkage-based approaches for detecting alleles of relatively small effect such those conferring a 2-fold or 4-fold increase in disease risk.

The TDT approach in general requires genotype data from parents and an affected child to examine whether there is any excess/under transmission of any haplotype. Transmitted haplotypes can be considered as cases and non-transmitted haplotypes can be considered as controls.

### EXAMPLE 3

### Transmission Disequilibrium Analysis of the Polymorphisms of the Invention

We conducted a family based association study on the NIMH schizophrenia data generated in Example 2. Disease status, data, pedigree structures and genotype data were analyzed with the computer program “Transmit”. Clayton, D. Am J Hum Genet, 1999. 65 (4): p.1170-7.

Description of the Data

There were 248 individuals (with genotype data of 225 individuals) from 62 nuclear families selected from NIMH schizophrenia collection for our study. There were 53 individual with unknown maternal ethnicity, roughly 68 individuals' mother's were African American, 51 were West European, 39 were Anglo-Saxon, 14 were Mediterranean and 23 with other ethnic background (numbers not entirely additive) A total of 111 individuals were diagnosed with schizophrenia, 50 individuals never had mental illness, 15 individuals had unknown disease statuses and 72 individuals had different types of mental illness.

Analytic Methods

To minimize the impact of admixture effect, a generalized transmission/disequilibrium tests (TDT) performed using program Transmit. It has been suggested that the type I errors of Transmit was not affected by population stratification. The p-value obtained from bootstrapping is reported. When one or both parent are not available for genotyping, Transmit utilizes the genotype data from extra children to infer parents' genotype.

Results

We performed “global tests” which examined whether haplotypes are transmitted to schizophrenia affected offspring more often than expected. For example, there are four haplotypes G-C, G-T, C—C, and C-T for the combination of SNP S1 and S4 of gene C202. We observed that haplotype G-C and G-T are transmitted to affected offspring more than expected and C—C, and C-T transmitted less than expected. The significant level of this observation is 0.000347 by bootstrapping.

Tests were also performed on individual haplotypes. The tests examined whether any haplotype is transmitted to affected offspring more often than other haplotypes. Again, for the SNP S1 and S4 of gene C202, we observed that the haplotype C—C is transmitted to affected offspring less than expected compared to other haplotypes combined. The significance of this observation is less than 0.0001 by bootstrapping, which is the most significant one in the all four individual haplotypes tests. All the combinations of selected SNPs were examined. There were several haplotypes of SNPs which showed significant association of these two genes. The test results of C202 are shown in Table 5.

TABLE 5

| | Global P- | | Observed | Observed | Expected | Expected | Individual P- |
|---|---|---|---|---|---|---|---|
| SNP loci | value | Haplotype | count | frequency | Count | Frequency | value |
| S1-S3 | 0.000299 | G-C | 166.07 | 0.8473 | 164.4 | 0.8388 | 0.34403 |
| | (0.00255) | | | | | | |
| | | C-C | 6.934 | 0.0354 | 11.334 | 0.0578 | <0.0001 |
| | | G-T | 22.934 | 0.117 | 19.68 | 0.1004 | 0.061571 |
| | | C-T | 0.065989 | 0.0003 | 0.58815 | 0.003 | <0.0001 |
| S1-S4 | 0.000357 | G-C | 168.07 | 0.8575 | 165.43 | 0.844 | 0.10928 |
| | (0.00035) | | | | | | |
| | | C-C | 6.934 | 0.0354 | 11.332 | 0.0578 | <0.0001 |
| | | G-T | 20.934 | 0.1068 | 18.651 | 0.0952 | 0.15797 |
| | | C-T | 0.065991 | 0.0003 | 0.58809 | 0.003 | <0.0001 |
| S1-S2 | 0.001964 | G-C | 148 | 0.7629 | 136.47 | 0.7035 | 0.00139 |
| | | C-C | 7 | 0.0361 | 11.83 | 0.061 | 0.008866 |
| | | G-A | 39 | 0.201 | 45.7 | 0.2356 | 0.046714 |
| S1 | 0.006932 | G | 189 | 0.9643 | 184.05 | 0.939 | 0.006932 |
| | | C | 7 | 0.0357 | 11.952 | 0.061 | 0.006932 |
| S7 | 0.020711 | A | 135 | 0.7105 | 126.32 | 0.6648 | 0.020711 |
| | | G | 55 | 0.2895 | 63.675 | 0.3351 | 0.020711 |
| S1-S7 | 0.025093 | G-A | 137.13 | 0.7069 | 127.87 | 0.6591 | 0.016123 |
| | | C-A | 1.1404 | 0.0059 | 1.257 | 0.0065 | 0.742 |
| | | G-G | 49.874 | 0.2571 | 54.208 | 0.2794 | 0.22338 |
| | | C-G | 5.8596 | 0.0302 | 10.667 | 0.055 | 0.001941 |
| S1-S2-S7 | 0.054154 | C-C-G | 137.94 | 0.711 | 128.71 | 0.6635 | 0.003 |
| | | G-C-A | 6 | 0.0309 | 10.767 | 0.0555 | 0.0116 |

As noted above, the global p values were calculated using Transmit. P values for individual haplotypes were also calculated. In addition the global p values for the most significant loci (S1-S3 and S1-S4) have been analyzed by an alternative method (results disclosed in parentheses in the table above).

We have done this by creating simulated genotype data which is irrelevant to NIMH phenotype data (disease status) for our NIMH pedigrees and analyzed the data with Transmit (Which is equivalent to analyzing the genotype data of SNP/SNPs which have nothing to do with the disease) and have determined that Transmit may tend to underestimate p values from this type of data. This phenomenon is more distinct for very small p values than for larger p values.

Briefly, our simulated genotype data for ten thousand pedigrees was generated with the population haplotype frequencies estimated from NIMH data under the assumption that Hardy-Weinberg Equilibrium and Mendel's First Law (equal segregation) are valid. Each pedigree with the phenotype data from NIMH and generated genotype was analyzed by Transmit and the results recorded. This process is similar to examining 10,000 genes in the genome that are unrelated to schizophrenia. The p-value reported by Transmit for the haplotype of S1-S3 of Con-202 is 0.000299. However, 22.5 out of 10,000 pedigrees has a p-value equal or less than 0.000299. Ideally the global p-values should be the same as the empirical p-values.

The meaning of the numerical value of a p-value is the probability that you have a result that is equal to or more extreme than the one we have simply by chance.

For the above example, we should observe about 3 out of 10,000 pedigrees (10,000×0.000299=2.99) to have a p-value equal to or smaller than 0.000299, but we observed 22.5 out of 10,000. Therefore, by this alternate method the p-value is reported to be 0.00255. Similarly the p-value for the S1-S4 haplotype can be reported as 0.0029 by similar analysis. This phenomenon is more distinct for very small p-values than larger p-values. When a reported p-value is about 0.05, the empirical p-value is fairly close to 0.05, therefore the S7, S1-S7, and S1-S2-S7 remain essentially unchanged by this refined analysis.

D. Statistical Methods

In general, any method known in the art to test whether a trait and a genotype show a statistically significant correlation may be used.

1. Methods in Linkage Analysis

Statistical methods and computer programs useful for linkage analysis are well-known to those skilled in the art (see Terwilliger J. D. and Ott J., Handbook of Human Genetic Linkage, John Hopkins University Press, London, 1994; Ott J., Analysis of Human Genetic Linkage, John Hopkins University Press, Baltimore, 1991).

2. Methods to Estimate Haplotype Frequencies in a Population

As described above, when genotypes are scored, it is often not possible to distinguish heterozygotes so that haplotype frequencies cannot be easily inferred. When the gametic phase is not known, haplotype frequencies can be estimated from the multilocus genotypic data. Any method known to person skilled in the art can be used to estimate haplotype frequencies (see Lange K., Mathematical and Statistical Methods for Genetic Analysis, Springer, New York, 1997; Weir, B. S., Genetic data Analysis I: Methods for Discrete population genetic Data, Sinauer Assoc., Inc., Sunderland, Mass. USA, 1996). Preferably, maximum-likelihood haplotype frequencies are computed using an Expectation-Maximization (EM) algorithm (see Dempster et al., J R. Stat. Soc., 39B: 1-38, 1977; Excoffier L. and Slatkin M., Mol. Biol. Evol., 12(5): 921-927, 1995). This procedure is an iterative process aiming at obtaining maximum-likelihood estimates of haplotype frequencies from multi-locus genotype data when the gametic phase is unknown. Haplotype estimations are usually performed by applying the EM algorithm using for example the EM-HAPLO program (Hawley M. E. et al., Am. J. Phys. Anthropol., 18:104, 1994) or the Arlequin program (Schneider et al., Arlequin: a software for population genetics data analysis, University of Geneva, 1997). The EM algorithm is a generalized iterative maximum likelihood approach to estimation and is briefly described below.

In the following part of this application, phenotypes will refer to multi-locus genotypes with unknown phase. Genotypes will refer to known-phase multi-locus genotypes.

Suppose a sample of N unrelated individuals typed for K markers. The data observed are the unknown-phase K-locus phenotypes that can categorized in F different phenotypes. Suppose that we have H underlying possible haplotypes (in case of K polymorphic markers, H=2^{k}). For phenotype j, suppose that Cⱼ genotypes are possible. We thus have the following equation:

$${P}_{j} = {\sum }_{i = 1}^{{c}_{j}} pr ( genotype i ) = {\sum }_{i = 1}^{{c}_{j}} pr ( {h}_{k} {h}_{l} ) \qquad Equation 1$$

where Pⱼ is the probability of the phenotype j, hₖ and hₗ, are the two haplotypes constituent in the genotype i. Under the Hardy-Weinberg equilibrium, pr(hₖ, hⱼ₎) becomes

pr(h^{k}, hₗ,)=pr(hₖ)²ifk=1, pr(hₖ, hₗ)=2pr(hₖ).pr(hₗ) if k≠l Equation 2

The successive steps of the E-M algorithm can be described as follows: Starting with initial values of the of haplotypes frequencies, noted p₁⁽⁰⁾,p⁽⁰⁾, . . . p_{H}⁽⁰⁾ these initial values serve to estimate the genotype frequencies (Expectation step) and then estimate another set of haplotype frequencies (Maximisation step), noted p₁⁽¹⁾,p⁽¹⁾, . . . p_{H}⁽¹⁾, these two steps are iterated until changes in the sets of haplotypes frequency are very small.

A stop criterion can be that the maximum difference between haplotype frequencies between two iterations is less than 10⁻⁷. These values can be adjusted according to the desired precision of estimations.

In details, at a given iteration s, the Expectation step consists in calculating the genotypes frequencies by the following equation:

$${P ( {h}_{k} , {h}_{l} )}^{( s )} = \frac{nj}{N} \cdot \frac{pr {( {h}_{k} , {h}_{l} )}^{( s )}}{{P}_{j}^{( s )}} \qquad Equation 3$$

where genotype I occurs in phenotype j, and where hₖ and hₗ, constitute genotype i. Each probability is derived according to equation 1, and equation 2 described above.

Then the Maximisation step simply estimates another set of haplotype frequencies given the genotypes frequencies. This approach is also known as the gene-counting method (Smith, Ann. Hum. Genet., 21:254-276, 1957).

$${P}_{t}^{( s + 1 )} = \frac{1}{2} {\sum }_{j = 1}^{F} {\sum }_{i = 1}^{{c}_{j}} {\partial }_{it} \cdot {pr ( genotype i )}^{( s )} \qquad Equation 4$$

Where ∂ᵢₜ, is an indicator variable which count the number of time haplotype t in genotype i. It takes the values of 0, 1 or 2.

To ensure that the estimation finally obtained is the maximum-likelihood estimation several values of departures are required. The estimations obtained are compared and if they are different the estimations leading to the best likelihood are kept.

3. Methods to Calculate Linkage Disequilibrium Between Markers

A number of methods can be used to calculate linkage disequilibrium between any two genetic positions, in practice linkage disequilibrium. is measured by applying a statistical association test to haplotype data taken from a population. Linkage disequilibrium between any pair of polymorphic markers comprising at least one of the polymorphic markers of the present invention (Mᵢ, Mⱼ) having alleles (aᵢ/bᵢ) at marker Mi and alleles (aⱼ/bⱼ) at marker Mj can be calculated for every allele combination (aᵢ,aⱼ; aᵢ,bⱼ; bᵢ,aⱼ and bᵢ,bⱼ), according to the Piazza formula: Δₐᵢₐⱼ=√04−√(04+03) (04+02), where:

04=−−frequency of genotypes not having allele aᵢ at Mᵢ and not having allele aⱼ at Mⱼ

03=−+frequency of genotypes not having allele aᵢ at Mᵢ and having allele aⱼ at Mⱼ

02=+−frequency of genotypes having allele aᵢ at Mᵢ and not having allele aⱼ at Mᵢ

Linkage disequilibrium (LD) between pairs of polymorphic markers (Mᵢ, Mⱼ) can also be calculated for every allele combination (aᵢ,aⱼ; aᵢ,bⱼ; bᵢ,aⱼ and bᵢ,bⱼ), according to the maximum-likelihood estimate (MLE) for delta (the composite genotypic disequilibrium coefficient), as described by Weir (Weir B. S., Genetic Data Analysis, Sinauer Ass. Eds, 1996). The MLE for the composite linkage disequilibrium is:

Dₐᵢₐⱼ=(2n₁, +n₂+n₃+n₄/2)/N−2(pr(aⱼ)·pr(aⱼ))

Where n₁=Σ phenotype (aᵢ/aᵢ, aⱼ/aⱼ), n2=Σphenotype (aᵢ/aᵢ, aⱼ/bⱼ), n3=Σphenotype (aᵢ/bᵢ, aⱼ/aⱼ), n4=Z phenotype (aᵢ/bᵢ, aⱼ/bⱼ) and N is the number of individuals in the sample. This formula allows linkage disequilibrium between alleles to be estimated when only genotype, and not haplotype, data are available.

Another means of calculating the linkage disequilibrium between markers is as follows. For a couple of polymorphic markers, Mᵢ(aᵢ bⱼ) and Mⱼ(aₗbⱼ), fitting the Hardy—

Weinberg equilibrium, one can estimate the four possible haplotype frequencies in a given population according to the approach described above.

The estimation of gametic disequilibrium between ai and aj is simply:

D'ₐᵢₐⱼ=pr(haplotype(aᵢ, aⱼ))−pr(aᵢ)−pr(aⱼ).

Where pr(aᵢ) is the probability of allele aᵢ and pr(aⱼ) is the probability of allele aj and where pr(haplotype (aᵢ, aⱼ)) is estimated as in Equation 3 above. For a couple of polymorphic markers only one measure of disequilibrium is necessary to describe the association between Mᵢ and Mⱼ.

Then a normalized value of the above is calculated as follows:

D'ₐ_{ᵢ}ₐ_{ⱼ}=D'ₐ_{ᵢ}ₐ_{ⱼ}/max(−pr(aᵢ).pr(aⱼ), −pr(bᵢ).pr(bⱼ)) with Dₐ_{ᵢ}ₐ_{ⱼ}<O

D'ₐ_{ᵢ}ₐ_{ⱼ}=D'ₐ_{ᵢ}ₐ_{ⱼ}/max(pr(bᵢ).pr(aⱼ), pr(aᵢ).pr(bⱼ)) with Dₐ_{ᵢ}ₐ_{ⱼ}>O

The skilled person will readily appreciate that other LD calculation methods can be used without undue experimentation. Linkage disequilibrium, among a set of polymorphic markers having an adequate heterozygosity rate can be determined by genotyping between 50 and 1000 unrelated individuals, preferably between 75 and 200, more preferably around 100.

4. Testing for Association

Methods for determining the statistical significance of a correlation between a phenotype and a genotype, in this case an allele at a polymorphic marker or a haplotype made up of such alleles, may be determined by any statistical test known in the art and with any accepted threshold of statistical significance being required. The application of particular methods and thresholds of significance are well with in the skill of the ordinary practitioner of the art.

Testing for association is performed by determining the frequency of a polymorphic marker allele in case and control populations and comparing these frequencies with a statistical test to determine if their is a statistically significant difference in frequency which would indicate a correlation between the trait and the polymorphic marker allele under study. Similarly, a haplotype analysis is performed by estimating the frequencies of all possible haplotypes for a given set of polymorphic markers in case and control populations, and comparing these frequencies with a statistical test to determine if their is a statistically significant correlation between the haplotype and the phenotype (trait) under study. Any statistical tool useful to test for a statistically significant association between a genotype and a phenotype may be used. Preferably the statistical test employed is a chi-square test with one degree of freedom. A P-value is calculated (the P-value is the probability that a statistic as large or larger than the observed one would occur by chance).

Statistical Significance

In preferred embodiments, significance for diagnosis purposes, either as a positive basis for further diagnostic tests or as a preliminary starting point for early preventive therapy, the p value related to a polymorphic marker association is preferably about 1×10⁻² or less, more preferably about 1×10⁻⁴ or less, for a single polymorphic marker analysis and about 1×10⁻³ or less, still more preferably 1×10⁻⁶ or less and most preferably of about 1×10⁻⁸ or less, for a haplotype analysis involving several markers. These values are believed to be applicable to any association studies involving single or multiple marker combinations.

The skilled person can use the range of values set forth above as a starting point in order to carry out association studies with polymorphic; markers of the present invention. In doing so, significant associations between the polymorphic markers of the present invention and CNS disorders can be revealed and used for diagnosis and drug screening purposes.

Phenotypic Permutation

In order to confirm the statistical significance of the first stage haplotype analysis described above, it might be suitable to perform further analyses in which genotyping data from case-control individuals are pooled and randomized with respect to the trait phenotype. Each individual genotyping data is randomly allocated to two groups, which contain the same number of individuals as the case-control populations used to compile the data obtained in the first stage. A second stage haplotype analysis is preferably run on these artificial groups, preferably for the markers included in the haplotype of the first stage analysis showing the highest relative risk coefficient. This experiment is reiterated preferably at least between 100 and 10000 times. The repeated iterations allow the determination of the percentage of obtained haplotypes with a significant p-value level.

Assessment of Statistical Association

To address the problem of false positives similar analysis may be performed with the same case-control populations in random genomic regions. Results in random regions and the candidate region are compared as described in WO 00/28080 entitled “Methods, software and apparati for identifying genomic regions harboring a gene associated with a detectable trait”.

Evaluation of Risk Factors

The association between a risk factor (in genetic epidemiology the risk factor is the presence or the absence of a certain allele or haplotype at marker loci) and a disease is measured by the odds ratio (OR) and by the relative risk (RR). If P(R⁺) is the probability of developing the disease for individuals with R and P(R7) is the probability for individuals without the risk factor, then the relative risk is simply the ratio of the two probabilities, that is: RR═P(R⁺)/P(R⁻)

In case-control studies, direct measures of the relative risk cannot be obtained because of the sampling design. However, the odds ratio allows a good approximation of the relative risk for low-incidence diseases and can be calculated:

OR=[F+/(1−F⁺]/[F⁻/(1−F⁻)]

F+is the frequency of the exposure to the risk factor in cases and F⁻ is the frequency of the exposure to the risk factor in controls. F+ and F⁻ are calculated using the allelic or haplotype frequencies of the study and further depend on the underlying genetic model (dominant, recessive, additive . . . ).

One can further estimate the attributable risk (AR) which describes the proportion of individuals in a population exhibiting a trait due to a given risk factor. This measure is important in quantitating the role of a specific factor in disease etiology and in terms of the public health impact of a risk factor. The public health relevance of this measure lies in estimating the proportion of cases of disease in the population that could be prevented if the exposure of interest were absent. AR is determined as follows:

AR=P_{E}(RR−I)/(P_{E}(RR−I)+1)

AR is the risk attributable to a polymorphic marker allele or a polymorphic marker haplotype. PE is the frequency of exposure to an allele or a haplotype within the population at large; and RR is the relative risk which, is approximated with the odds ratio when the trait under study has a relatively low incidence in the general population.

Association of Polymorphic Markers of the Invention with Schizophrenia

In the context of the present invention, an association between polymorphic marker alleles of the present invention in Con-202 and schizophrenia was demonstrated

Many neurochemical findings are coming to light implicating a biological basis for schizophrenia, at least for certain subtypes. However, the lack of a defined and specific schizophrenia phenotype and of suitable markers for genetic analysis is proving to be a major hurdle for reliably identifying genes associated with schizophrenia. As a result, psychiatrists today have to choose anti-schizophrenia medications by intuition and trial and error; a situation that can put suicidal patients in jeopardy for weeks or months until the right compound is selected. Clearly, there is a strong need to successfully identify genes involved in schizophrenia; thus allowing researchers to understand the etiology of depression and address its cause, rather than symptoms.

This information is extremely valuable. The knowledge of a potential genetic predisposition, even if this predisposition is not absolute, might contribute in a very significant manner to treatment efficacy of depressed patients and to the development of diagnostic tools.

It will be clear that the invention may be practiced otherwise than as particularly described in the foregoing description and examples.

Numerous modifications and variations of the present invention are possible in light of the above teachings and, therefore, are within the scope of the invention

The entire disclosures of all publications cited herein are hereby incorporated by reference.

What is claimed is:

1. An isolated polynucleotide consisting of between 12 and 200 contiguous nucleotides of SEQ ID NO 1 or its complement including at least one Con-202 polymorphic site selected from the group consisting of the polymorphic sites at positions 184, 1022, 1292, 1400, 2296, 2369 and 2724.

2. The isolated polynucleotide of claim 1 in which the nucleotide at position 184 is selected from the group of nucleotides G or C

3. The isolated polynucleotide which is the complement of the isolated polynucleotide of claim 2.

4. The isolated polynucleotide of claim 1 in which the nucleotide at position 1022 is selected from the group of nucleotides C or A

5. The isolated polynucleotide which is the complement of the isolated polynucleotide of claim 4.

6. The isolated polynucleotide of claim 1 in which the nucleotide at position 1292 is selected from the group of nucleotides C or T.

7. The isolated polynucleotide which is the complement of the isolated polynucleotide of claim 6.

8. The isolated polynucleotide of claim 1 in which the nucleotide at position 1400 is selected from the group of nucleotides C or T.

9. The isolated polynucleotide which is the complement of the isolated polynucleotide of claim 8.

10. The isolated polynucleotide of claim 1 in which the nucleotide at position 2296 is selected from the group of nucleotides T or A.

11. The isolated polynucleotide which is the complement of the isolated polynucleotide of claim 10.

12. The isolated polynucleotide of claim 1 in which the nucleotide at position 2369 is selected from the group of nucleotides G or A.

13. The isolated polynucleotide which is the complement of the isolated polynucleotide of claim 12.

14. The isolated polynucleotide of claim 1 in which the nucleotide at position 2724 is selected from the group of nucleotides A or G.

15. The isolated polynucleotide which is the complement of the isolated polynucleotide of claim 14.

16. The isolated polynucleotide of claim 1 that is less than 200 nucleotides

17. The isolated polynucleotide of claim 1 that is less than 50 nucleotides.

18. The isolated polynucleotide of claim 1 that is less than 20 nucleotides.

19 The isolated polynucleotide of claim 1 wherein the polymorphism is within 4 nucleotides of the center of said polynucleotide.

20. The isolated polynucleotide of claim 1 wherein the polymorphism is at the center of said polynucleotide.

21. The isolated polynucleotide of claim 1 wherein the polymorphism is at the end of said polynucleotide.

22. The isolated polynucleotide of claim 1 wherein the polynucleotide is a probe.

23. The isolated polynucleotide of claim 1 wherein the polynucleotide is a primer.

24. An isolated polynucleotide comprising at least 12 contiguous nucleotides of SEQ ID NO:1 or its complement including at least one Con-202 polymorphic site selected from the group consisting of the Con-202 polymorphic sites at positions 184, 1022, 2296 and 2369 wherein said Con-202 polymorphic site or sites is occupied by at least one rare allele selected from the group consisting of C at position 184, A at position 1022, A at position 2296 and A at position 2369.

25. An isolated polynucleotide according to claim 24 comprising at least 12 contiguous nucleotides of SEQ ID NO:1 including the nucleotide at position 184 wherein position 184 is occupied by a C.

26. The isolated polynucleotide which is the complement of the isolated polynucleotide of claim 25.

27. An isolated polynucleotide according to claim 24 comprising at least 12 contiguous nucleotides of SEQ ID NO:1 including the nucleotide at position 1022 wherein position 1022 is occupied by an A.

28. The isolated polynucleotide which is the complement of the isolated polynucleotide of claim 27.

29. An isolated polynucleotide according to claim 24 comprising at least 12 contiguous nucleotides of SEQ ID NO:1 including the nucleotide at position 2296 wherein position 2296 is occupied by an A.

30. The isolated polynucleotide which is the complement of the isolated polynucleotide of claim 29.

31. An isolated polynucleotide according to claim 24 comprising at least 12 contiguous nucleotides of SEQ ID NO:1 including the nucleotide at position 2369 wherein position 2369 is occupied by an A.

32. The isolated polynucleotide which is the complement of the isolated polynucleotide of claim 31.

33. A method for classifying a nucleic acid molecule encoding Con-202 or a fragment thereof from an individual comprising
(a) obtaining said nucleic acid from said individual; and
(b) determining a nucleotide from said nucleic acid which corresponds to the nucleotide occupying at least one polymorphic site selected from the group consisting of the polymorphic sites at positions 184, 1022, 1292, 1400, 2296, 2369 and 2724

34. The method of claim 33, wherein the determining comprises determining whether the nucleotide occupying position 184 of SEQ ID NO:1 or a fragment thereof is a G

35. The method of claim 33, wherein the determining comprises determining whether the nucleotide occupying position 184 of SEQ ID NO:1 or a fragment thereof is a C

36. The method of claim 33, wherein the determining comprises determining whether the nucleotide occupying position 1022 of SEQ ID NO:1 or a fragment thereof is a C

37. The method of claim 33, wherein the determining comprises determining whether the nucleotide occupying position 1022 of SEQ ID NO:1 or a fragment thereof is an A.

38. The method of claim 33, wherein the determining comprises determining whether the nucleotide occupying position 1292 of SEQ ID NO:1 or a fragment thereof is a C.

39. The method of claim 33, wherein the determining comprises determining whether the nucleotide occupying position 1292 of SEQ ID NO:1 or a fragment thereof is a T.

40 The method of claim 33, wherein the determining comprises determining whether the nucleotide occupying position 1400 of SEQ ID NO:1 or a fragment thereof is a C

41. The method of claim 33, wherein the determining comprises determining whether the nucleotide occupying position 1400 of SEQ ID NO:1 or a fragment thereof is a T

42. The method of claim 33, wherein the determining comprises determining whether the nucleotide occupying position 2724 of SEQ ID NO:1 or a fragment thereof is an A

43. The method of claim 33, wherein the determining comprises determining whether the nucleotide occupying position 2724 of SEQ ID NO:1 or a fragment thereof is a G

44. The method of claim 33, wherein the determining comprises amplification of the DNA.

45. A method of haplotype determination in an individual for diagnostic or prognostic purposes, comprising
obtaining said nucleic acid from said individual; and
determining the nucleotides from said nucleic acid which correspond to the nucleotides occupying more than one Con-202 polymorphic site selected from the group consisting of the Con-202 polymorphic sites at positions:
(a) 184 and 1292,
(b) 184 and 1400,
(c) 184 and 1022,
(c) 184 and 2724 and
(d) 184 and 1022 and 2724
on a single chromosome.

46. The method of claim 45, wherein the determining comprises determining whether the nucleotide occupying position 184 and 1292 of SEQ ID NO:1 or a fragment thereof are both C.

47. The method of claim 45, wherein the determining comprises determining whether the nucleotide occupying position 184 of SEQ ID NO:1 or a fragment thereof is a C and whether the nucleotide occupying position 1292 of SEQ ID NO:1 or a fragment thereof is a T.

48. The method of claim 45, wherein the determining comprises determining whether the nucleotide occupying position 184 and 1400 of SEQ ID NO:1 or a fragment thereof are both C.

49. The method of claim 45, wherein the determining comprises determining whether the nucleotide occupying position 184 of SEQ ID NO:1 or a fragment thereof is a C and whether the nucleotide occupying position 1400 of SEQ ID NO:1 or a fragment thereof is a T.

50. The method of claim 45, wherein the determining comprises determining whether the nucleotide occupying position 184 and 1022 of SEQ ID NO:1 or a fragment thereof are both C.

51. The method of claim 45, wherein the determining comprises determining whether the nucleotide occupying position 184 of SEQ ID NO:1 or a fragment thereof is a G and whether the nucleotide occupying position 1022 of SEQ ID NO:1 or a fragment thereof is a C.

52. The method of claim 45, wherein the determining comprises determining whether the nucleotide occupying position 184 of SEQ ID NO:1 or a fragment thereof is a G and whether the nucleotide occupying position 1022 of SEQ ID NO:1 or a fragment thereof is a A.

53. The method of claim 45, wherein the determining comprises determining whether the nucleotide occupying position 184 of SEQ ID NO:1 or a fragment thereof is a C and whether the nucleotide occupying position 2724 of SEQ ID NO:1 or a fragment thereof is a G.

54. The method of claim 45, wherein the determining comprises determining whether the nucleotide occupying position 184 of SEQ ID NO:1 or a fragment thereof is a G and whether the nucleotide occupying position 2724 of SEQ ID NO:1 or a fragment thereof is a A.

55. The method of claim 45, wherein the determining comprises:
determining whether the nucleotide occupying position 184 of SEQ ID NO:1 or a fragment thereof is a C,
determining whether the nucleotide occupying position 1022 of SEQ ID NO:1 or a fragment thereof is a C and
determining whether the nucleotide occupying position 2724 of SEQ ID NO:1 or a fragment thereof is a G.

56. The method of claim 45, wherein the determining comprises:
determining whether the nucleotide occupying position 184 of SEQ ID NO:1 or a fragment thereof is a G,
determining whether the nucleotide occupying position 1022 of SEQ ID NO:1 or a fragment thereof is a C and
determining whether the nucleotide occupying position 2724 of SEQ ID NO:1 or a fragment thereof is an A.

57. The method of claim 45, wherein the determining comprises amplification of the DNA

58. The method of claim 30 wherein the determining comprises double PCR amplification of specific alleles.

59. A method of predicting a whether an individual is more or less likely to suffer from schizophrenia comprising:
determining the nucleotide from a nucleic acid sample from said patient which corresponds to the nucleotide occupying a Con-202 polymorphic site selected from the group consisting of the Con-202 polymorphic sites at positions: 184 and 2724.

60. The method of claim 43 wherein the presence of a G at position 184 indicates that the individual is more likely to suffer from schizophrenia.

61. The method of claim 43 wherein the presence of a C at position 184 indicates that the individual is less likely to suffer from schizophrenia.

62. The method of claim 43 wherein the presence of an A at position 2724 indicates that the individual is more likely to suffer from schizophrenia.

63. The method of claim 43 wherein the presence of an G at position 2724 indicates that the individual is less likely to suffer from schizophrenia.

64. A method of predicting a whether an individual is more or less likely to suffer from schizophrenia comprising:
determining the nucleotides from a nucleic acid sample from said patient which correspond to two or more nucleotides occupying two or more Con-202 polymorphic sites on a single chromosome selected from the group consisting of the Con-202 polymorphic sites at positions:
(a) 184 and 1292,
(b) 184 and 1400,
(c) 184 and 1022,
(c) 184 and 2724 and
(d) 184 and 1022 and 2724

65. The method of claim 64 wherein a C at both positions 184 and 1292 indicates that the individual is less likely to suffer from schizophrenia.

66. The method of claim 64 wherein a C at position 184 and a T at position 1292 indicates that the individual is less likely to suffer from schizophrenia.

67. The method of claim 66 wherein a C at both positions 184 and 1400 indicates that the individual is less likely to suffer from schizophrenia.

68. The method of claim 66 wherein a C at position 184 and a T at position 1400 indicates that the individual is less likely to suffer from schizophrenia.

69. The method of claim 66 wherein a G at position 184 and a C at position 1022 indicates that the individual is more likely to suffer from schizophrenia.

70. The method of claim 66 wherein a C at both positions 184 and 1022 indicates that the individual is less likely to suffer from schizophrenia.

71. The method of claim 66 wherein a G at position 184 and an A at position 1022 indicates that the individual is less likely to suffer from schizophrenia.

72. The method of claim 66 wherein a G at position 184 and an A at position 2724 indicates that the individual is more likely to suffer from schizophrenia.

73. The method of claim 66 wherein a G at both positions 184 and 2724 indicates that the individual is less likely to suffer from schizophrenia.

74. The method of claim 66 wherein a C at position 184 and an G at position 2724 indicates that the individual is less likely suffer from schizophrenia.

75. The method of claim 66 wherein a C at both positions 184 and 1022 and a G at position 2724 indicates that the individual is more likely to suffer from schizophrenia.

76. The method of claim 66 wherein a G at position 184, a C at position 1022, and an A at position 2724 indicates that the individual is less likely to suffer from schizophrenia.

77. A method of determining the frequency in a population of an Con-202 polymorphism, comprising:
a) genotyping individuals from said population for said polymorphism according to the method of claim 33; and
b) determining the proportional representation of said polymorphism in said population.

78. The method of detecting an association between a Con-202 polymorphism and a phenotype, comprising the steps of:
a) determining the frequency of at least one Con-202 polymorphism in a trait positive population according to the method of claim 77;
b) determining the frequency of said Con-202 polymorphism in a control population according to the method of claim 77; and
c) determining whether a statistically significant association exists between said polymorphism and said phenotype.

79. The method according to claim 78 wherein said phenotype is response to an agent acting on schizophrenia

80. The method according to claim 78 wherein said phenotype is a side effect to an agent acting on schizophrenia

81. A method of estimating the frequency of a haplotype for a set of polymorphic markers in a population, comprising:
a) genotyping each individual in said population for at least one Con-202 polymorphism according to claim 33;
b) genotyping each individual in said population for a second polymorphic marker by determining the identity of the nucleotides at said second polymorphic marker for both copies of said second polymorphic marker present in the genome; and
c) applying a haplotype determination method to the identities of the nucleotides determined in steps a) and b) to obtain an estimate of said frequency.

82. The method according to claim 81, wherein said haplotype determination method is selected from the group consisting of asymmetric PCR amplification, double PCR amplification of specific alleles, the Clark method, the Clayton method, or an expectation maximization algorithm.

83. A diagnostic kit comprising the required components for the determination of the of the identity of the nucleotide occupying a Con-202 polymorphic site in small volumes in a self contained kit.

84. The diagnostic kit of claim 83 comprising an isolated polynucleotide comprising between 12 and 200 contiguous nucleotides of SEQ ID NO 1 or its complement including at least one polymorphic site.
